(19) United States
(12) Patent Application Publication
(10) Pub. No.: US 2024/0122978 A1
(43) Pub. Date: Apr. 18, 2024

(54) RETROVIRAL VECTOR FOR UNIVERAL RECEPTOR THERAPY

(71) Applicant: Umoja BioPharma, Inc., Seattle, WA (US)

(72) Inventor: Andrew SCHARENBERG, Seattle, WA (US)

(21) Appl. No.: 17/766,654

(22) PCT Filed: Oct. 15, 2020
(86) PCT No.: PCT/US2020/055826

§ 371 Date: Apr. 5, 2022

Related U.S. Application Data
(60) Provisional application No. 62/916,110, filed on Oct. 16, 2019.

(51) Int. Cl. A61K 35/17; A61K 39/00; A61K 47/64; C07K 14/005; C07K 16/28; C12N 7/00; C12N 15/86

(52) U.S. Cl. CPC A61K 35/17; A61K 39/4611; A61K 39/4631; A61K 39/464429; A61K 47/643; A61K 47/646; C07K 14/005; C07K 16/2809; C07K 16/2827; C07K 16/2878; C12N 7/00; C12N 15/86; A61K 2039/505; C12N 2740/00022; C12N 2740/00033; C12N 2740/15022; C12N 2740/15043

(57) ABSTRACT

The present disclosure relates to retroviral vectors comprising polynucleotides encoding hapten-binding receptors and T cell activating receptors. The retroviral vectors may also comprise transduction enhancers. Also disclosed are adaptor molecules and their use in conjunction with the retroviral vectors and T cells transduced with the retroviral vectors.

### RELATED APPLICATIONS

This application claims priority to and the benefit of U.S. Provisional Application No. 62/916,110 filed Oct. 16, 2019, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Oct. 15, 2020, is named “UMOJ-003-01WO_SeqList ST25.txt” and is 72 KB in size.

### TECHNICAL FIELD

The present disclosure relates to viral vectors encoding transduction enhancers, synthetic T cell activating receptors, and/or hapten-binding receptors, compositions comprising, and methods of use thereof. The present disclosure also relates to the use of adaptor molecules comprising haptens for use in conjunction with the viral vectors. The disclosed compositions and methods may be useful in the treatment of disease, e.g., cancer.

### BACKGROUND

Chimeric antigen receptors (CARs) are engineered receptors used to genetically engineer T cells for use in adoptive cellular immunotherapy (see Pule et al., Cytother. 5:3, 2003; Restifo et al., Nat. Rev. Immunol. 12:269, 2012). These receptors include an extracellular ligand binding domain, most commonly a single chain variable fragment of a monoclonal antibody (scFv), linked to intracellular signaling components, most commonly CD3 alone or combined with one or more costimulatory domains. Antigen binding stimulates the signaling domains on the intracellular segment of the CAR, thereby activating signaling pathways. CAR-based adoptive cellular immunotherapy has been used to treat cancer patients with tumors refractory to conventional standard-of-care treatments (see Grupp et al., N Engl. J. Med. 368:1509, 2013; Kalos et al., Sci. Transl. Med. 3:95ra73, 2011).

Autologous T cells can be genetically modified to express transgenes that are engineered to enhance efficacy after transfer in vivo. Despite successful adoptive transfer of transgene modified T cells in the setting of, e.g., CD19 B cell lineage malignancies, no universal CAR target antigen that is present on all forms of cancer, but not normal, cells has been identified. Thus, the field is hampered by the need to identify cell surface targets that are naturally present on tumor cells and minimally expressed by normal cells/tissues of the body. Thus, CAR T cell therapeutic development is hampered by the prospect of potentially needing tens to hundreds of vetted CAR targets and CARs to cover the majority of cancer types afflicting humans. In addition, existing CAR technologies often require expensive and time-consuming ex vivo manipulation steps in order to activate and transduce T cells with CAR vectors prior to re-infusing hosts with CAR T cells. Furthermore CAR T cells can have numerous off-target effects that can lead to dangerous, even lethal adverse events.

There is an unmet need for safer, more cost-effective, and more universal CAR T cell technologies.

### SUMMARY

The disclosure relates generally to methods involving (a) administering to a subject an adaptor molecule comprising a targeting moiety and a hapten; and (b) administering to the subject either (i) a plurality of recombinant retroviral particles or (ii) cells produced by ex vivo contacting immune cells with a plurality of recombinant retroviral particles. Each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR. Each of the retroviral particles comprises a viral envelope. In some embodiments, the method is a method of treating cancer in a subject in need thereof. In some embodiments, the method is a method of killing tumor cells in a subject in need thereof.

In another aspect, the disclosure provides a system or composition including (a) an adaptor molecule comprising a targeting moiety and a hapten; and (b) either (i) a plurality of recombinant retroviral particles or (ii) cells produced by ex vivo contacting immune cells with a plurality of recombinant retroviral particles. Each of the retroviral particles comprises a polynucleotide comprising, in 5′ to 3′ order: (i) a 5′ long terminal repeat (LTR) or UTR, (ii) a promoter, (iii) a sequence encoding a receptor that specifically binds to the hapten, and (iv) a 3′ LTR or UTR. Each of the retroviral particles comprises a viral envelope. In some embodiments, the system or composition is provided in a kit including instruction for use thereof.

Further aspects and embodiments of the invention are provided by the Detailed Description that follows.

### BRIEF DESCRIPTION OF THE DRAWINGS

FIGS. 1A-1D are flow cytometry staining plots depicting surface expression of FITC-CAR constructs in transduced T cells. FIG. 1A depicts a flow cytometry staining plot of lymphocytes which were further gated to visualize single cells (FIG. 1B). The single cells were further gated to visualize CD3+ cells (FIG. 1C). The CD3+ cells were further gated to visualize FITC-CAR+cells (FIG. 1D).

FIGS. 2A-2F are flow cytometry staining plots depicting how transduced T cells expressing the RACR-CAR payload are enriched by the addition of rapamycin to activate the RACR (FIG. 2A and FIG. 2D), and/or are enriched by the addition of FITC antigen (FIG. 2B-2C, and FIG. 2E-2F) to activate the CAR. FIGS. 2A-2C depict transduced cells that were treated with 0 nM rapamycin and FIGS. 2D-2F depict transduced cells that were treated with 10 nM rapamycin. FIGS. 2B and 2E depict transduced cells that were stimulated with FITC coated beads and FIGS. 2C and 2F depict transduced cells that were stimulated with plate bound FITC.

FIG. 3 is a graph depicting the percentage of live FITC-CAR+T cells in transduced cells which were unstimulated, stimulated with FITC coated beads, or stimulated with platebound FITC, and with (10 nM) or without (0 nM) rapamycin.

FIG. 4 is a graph depicting the total number of live FITC-CAR+T cells in transduced cells which were unstimulated, stimulated with FITC coated beads, or stimulated with platebound FITC, and with (10 nM) or without (0 nM) rapamycin.

FIG. 5 is a graph depicting the upregulation of activation marker CD25 in transduced cells which were unstimulated, stimulated with FITC coated beads, or stimulated with platebound FITC, and with (10 nM) or without (0 nM) rapamycin. gMFI=geometric mean fluorescence intensity.

FIG. 6 depicts a vector map for FITC CAR-Frb-RACR.

FIG. 7 depicts a vector map for the CD3-Cocal viral particle envelope.

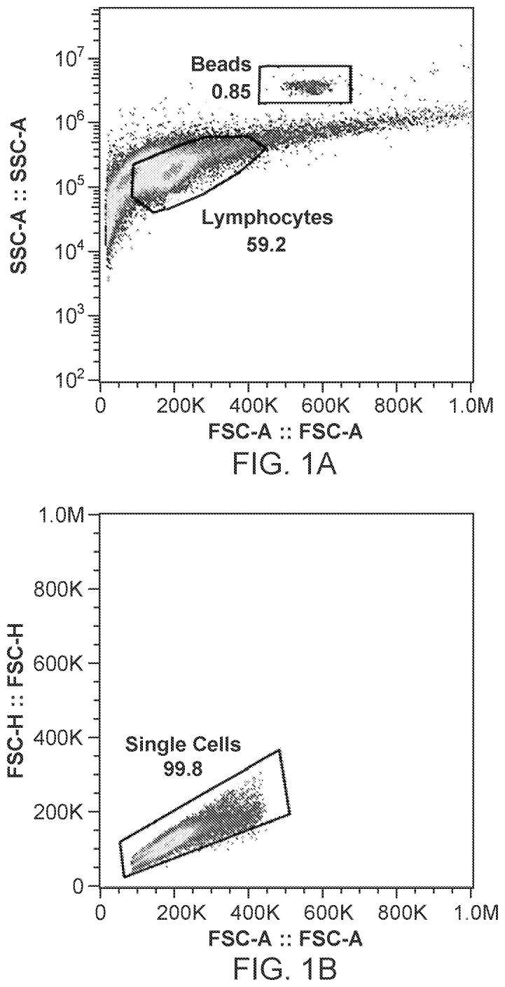

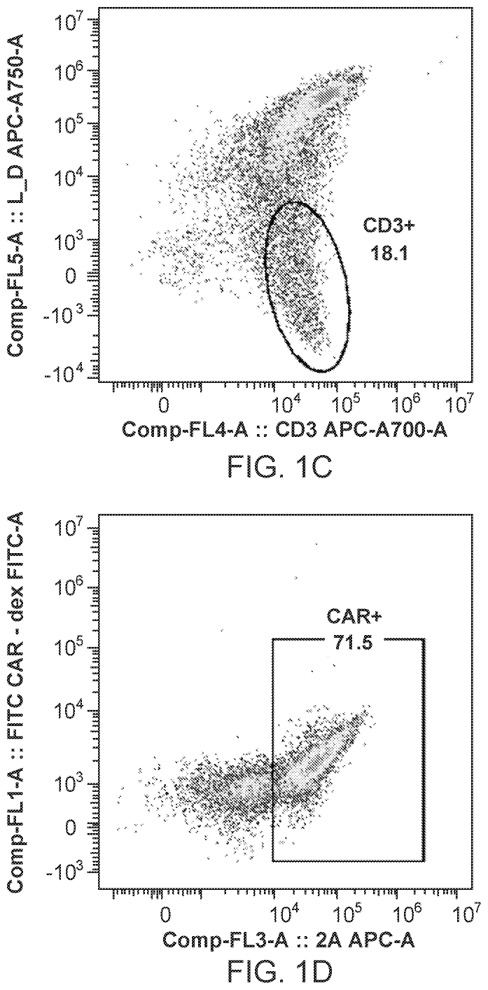

### DETAILED DESCRIPTION

The disclosure relates generally to a method involving (a) administering to a subject an adaptor molecule comprising a targeting moiety and a hapten; and (b) administering to the subject either (i) a plurality of recombinant retroviral particles or (ii) cells produced by ex vivo contacting immune cells with a plurality of recombinant retroviral particles. Each of the retroviral particles comprises a polynucleotide comprising a sequence encoding a receptor that specifically binds to the hapten. Each of the retroviral particles comprises a viral envelope. In some embodiments, the method is a method of treating cancer in a subject in need thereof. In some embodiments, the method is a method of killing tumor cells in a subject in need thereof.

In another aspect, the disclosure provides a system or composition including (a) an adaptor molecule comprising a targeting moiety and a hapten; and (b) either (i) a plurality of recombinant retroviral particles or (ii) cells produced by ex vivo contacting immune cells with a plurality of recombinant retroviral particles. Each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR. Each of the retroviral particles comprises a viral envelope. In some embodiments, the system or composition is provided in a kit including instruction for use thereof.

### Retroviral Particles

Retroviruses include lentiviruses, gamma-retrovirues, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, e.g. p7, p24, and p17, containing a retroviral or lentiviral envelope including one or more of the env encoded glycoproteins, e.g. p41, p120, and p160, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the hapten. In some embodiments, a sequence encoding a receptor that specifically binds to the hapten is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a hapten-binding receptor. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR.

In some embodiments, the retroviral particles comprise a cell surface receptor that binds to a ligand on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof. In some embodiments, the viral fusion glycoprotein comprises the amino acid sequence of SEQ ID NO: 1 (Cocal G protein). In some embodiments, the viral fusion glycoprotein comprises an amino acid sequence at least 95% identical to SEQ ID NO:1 (Cocal G protein). In some embodiments, the viral fusion glycoprotein comprises an amino acid sequence at least 50%, at least 55%, at least 60%, at least 65%, at least 70%, at least 75%, at least 80%, at least 85%, at least 90%, at least 95%, at least 96%, at least 97%, at least 98%, or at least 99% identical to SEQ ID NO: 1 (Cocal G protein).

(SEQ ID NO: 1)
NFLLLTFIVLPLCSHAKFSIVFPQSQKGNWKNVPSSYHYCPSSSDQNWHN
DLLGITMKVKMPKTHKAIQADGWMCHAAKWITTCDFRWYGPKYITHSIHS
IQPTSEQCKESIKQTKQGTWMSPGFPPQNCGYATVTDSVAVVVQATPHHV
LVDEYTGEWIDSQFPNGKCETEECETVHNSTVWYSDYKVTGLCDATLVDT
EITFFSEDGKKESIGKPNTGYRSNYFAYEKGDKVCKMNYCKHAGVRLPSG
VWFEFVDQDVYAAAKLPECPVGATISAPTQTSVDVSLILDVERILDYSLC
QETWSKIRSKQPVSPVDLSYLAPKNPGTGPAFTIINGTLKYFETRYIRID
IDNPIISKMVGKISGSQTERELWTEWFPYEGVEIGPNGILKTPTGYKFPL
FMIGHGMLDSDLHKTSQAEVFEHPHLAEAPKQLPEEETLFFGDTGISKNP
VELIEGWFSSWKSTVVTFFFAIGVFILLYVVARIVIAVRYRYQGSNNKRI
YNDIEMSRFRK

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors.

### Transduction Enhancers

In some embodiments, viral particles according to the present disclosure comprise transduction enhancers.

A “transduction enhancer” as used herein refers to a transmembrane protein that activates T cells. Transduction enhancers may be incorporated into the viral envelopes of viral particles according to the present disclosure. The transduction enhancer may comprise a mitogenic and/or cytokine-based domain. The transduction enhancer may comprise T cell activation receptors, NK cell activation receptors, co-stimulatory molecules, or portions thereof.

### Mitogenic Transduction Enhancers

The viral vector of the present invention may comprise a mitogenic transduction enhancer in the viral envelope. In some embodiments, the mitogenic transduction enhancer is derived from the host cell during retroviral vector production. In some embodiments, the mitogenic transduction enhancer is made by the packaging cell and expressed at the cell surface. When the nascent retroviral vector buds from the host cell membrane, the mitogenic transduction enhancer may be incorporated in the viral envelope as part of the packaging cell-derived lipid bilayer.

In some embodiments, the transduction enhancer is host-cell derived. The term “host-cell derived” indicates that the mitogenic transduction enhancer is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. In some embodiments, the packaging-cell derived mitogenic transduction enhancer of the present invention does not comprise the surface envelope subunit (SU).

The mitogenic transduction enhancer may have the structure: M-S-TM, in which M is a mitogenic domain; S is an optional spacer domain and TM is a transmembrane domain.

### Transduction Enhancer Mitogenic Domains

The mitogenic domain is the part of the mitogenic transduction enhancer which causes T-cell activation. It may bind or otherwise interact, directly or indirectly, with a T cell, leading to T cell activation. In particular, the mitogenic domain may bind a T cell surface antigen, such as CD3, CD28, CD134 and CD137.

CD3 is a T-cell co-receptor. It is a protein complex composed of four distinct chains. In mammals, the complex contains a CD3y chain, a CD35 chain, and two CD3e chains. These chains associate with the T-cell receptor (TCR) and the ζ-chain to generate an activation signal in T lymphocytes. The TCR, ζ-chain, and CD3 molecules together comprise the TCR complex.

In some embodiments, the mitogenic domain may bind to a CD3 ε chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD134, also known as OX40, is a member of the TNFR-superfamily of receptors which is not constitutively expressed on resting naive T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

The mitogenic domain may comprise the binding domain from OKT3, 15E8, TGN1412, CD28.2, 10F3, UCHT1, YTH12.5 or TR66.

The mitogenic domain may comprise all or part of a co-stimulatory molecule such as OX40L and 41 BBL. For example, the mitogenic domain may comprise the binding domain from OX40L or 41 BBL.

OKT3, also known as Muromonab-CD3 is a monoclonal antibody targeted at the CD3e chain. It is clinically used to reduce acute rejection in patients with organ transplants. It was the first monoclonal antibody to be approved for clinical use in humans. The CDRs of OKT3 are as follows1

CDRH1:
(SEQ ID NO. 4)
GYTFTRY
CDRH2:
(SEQ ID NO. 5)
NPSRGY
CDRH3:
(SEQ ID NO. 6)
YYDDHYCLDY
CDRL1:
(SEQ ID NO. 7)
SASSSVSYMN
CDRL2:
(SEQ ID NO. 8)
DTSKLAS
CDRL3:
(SEQ ID NO. 9)
QQWSSNPFT

15E8 is a mouse monoclonal antibody to human CD28. Its CDRs are as follows:

CDRH1:
(SEQ ID NO. 10)
GFSLTSY
CDRH2:
(SEQ ID NO. 11)
WAGGS
CDRH3:
(SEQ ID NO. 12)
DKRAPGKLYYGYPDY
CDRL1:
(SEQ ID NO. 13)
RASESVEYYVTSLMQ
CDRL2:
(SEQ ID NO. 14)
AASNVES
CDRL3:
(SEQ ID NO. 15)
QQTRKVPST

TGN1412 (also known as CD28-SuperMAB) is a humanised monoclonal antibody that not only binds to, but is a strong agonist for, the CD28 receptor. Its CDRs are as follows.

CDRH1:
(SEQ ID NO. 16)
GYTFSY
CDRH2:
(SEQ ID NO. 17)
YPGNVN
CDRH3:
(SEQ ID NO. 18)
SHYGLDWNFDV
CDRL1:
(SEQ ID NO. 19)
HASQNIYVLN
CDRL2:
(SEQ ID NO. 20)
KASNLHT
CDRL3:
(SEQ ID NO. 21)
QQGQTYPYT

OX40L is the ligand for CD134 and is expressed on such cells as DC2s (a subtype of dendritic cells) enabling amplification of Th2 cell differentiation. OX40L has also been designated CD252 (cluster of differentiation 252).

OX40L sequence
(SEQ ID NO. 22)
MERVQPLEENVGNAARPRFERNKLLLVASVIQGLGLLLCFTYICLHFSAL
QVSHRYPRIQSIKVQFTEYKKEKGFILTSQKEDEIMKVQNYLISLKGYFS
QEVNISLHYQKDEEPLFQLKKVRSVNSLMVASLTYKDKVYLNVTTDNTSL
DDFHVNGGELILIHQNPGEFCVL

4-1BBL is a cytokine that belongs to the tumour necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

41 BBL sequence
(SEQ ID NO. 23)
MEYASDASLDPEAPWPPAPRARACRVLPWALVAGLLLLLLLAAACAVFL
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFG
FQGRLLHLSAGQRLGVHLHTEARARHAWQLTQGATVLGLFRVTPEIPAGL
PSPRSE

### Transduction Enhancer Spacer Domains

The mitogenic transduction enhancer and/or cytokine-based transduction enhancer may comprise a spacer sequence to connect the antigen-binding domain with the transmembrane domain. A flexible spacer allows the antigen-binding domain to orient in different directions to facilitate binding.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an lgG1 Fc region, an lgG1 hinge or a CD8 stalk. A human lgG1 spacer may be altered to remove Fc binding motifs.

Examples of amino acid sequences for these spacers are given below:

SEQ ID NO. 24 (hinge-CH2CH3 of human lgG1)
AEPKSPDKTHTCPPCPAPPVAGPSVFLFPPKPKDTLMIARTPEVTCWVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGKKD
SEQ ID NO. 25 (human CD8 stalk):
TTTPAPRPPTPAPTIASQPLSLRPEACRPAAGGAVHTRGLDFACDI
SEQ ID NO. 26 (human lgG1 hinge):
AEPKSPDKTHTCPPCPKDPK
SEQ ID NO. 27 (CD2 ectodomain):
KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
SEQ ID NO. 28 (CD34 ectodomain):
SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

In some embodiments, the spacer sequence may be derived from a human protein.

### Transduction Enhancer Transmembrane Domains

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

An alternative option to a transmembrane domain is a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

### Cytokine-Based Transduction Enhancers

The viral vector of the present invention may comprise a cytokine-based transduction enhancer in the viral envelope. In some embodiments, the cytokine-based transduction enhancer is derived from the host cell during viral vector production. In some embodiments, the cytokine-based transduction enhancer is made by the host cell and expressed at the cell surface. When the nascent viral vector buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain and TM is the transmembrane domain. The spacer domain and transmembrane domains are as defined above.

### Transduction Enhancer Cytokine Domains

The cytokine domain may comprise part or all of a T-cell activating cytokine, such as from IL2, IL7 and IL15. The cytokine domain may comprise part of the cytokine, as long as it retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding. The sequence of IL2 is shown as SEQ ID NO. 29.

SEQ ID NO. 29:
MYRMQLLSCIALSLALVINSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

IL7 is a cytokine that serves as a growth factor for early lymphoid cells of both B-and T-cell lineages. The sequence of IL7 is shown as SEQ ID NO. 30.

SEQ ID NO. 30:
MFHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEH

IL15 is a cytokine with structural similarity to IL-2. Like IL-2, IL-15 binds to and signals through a complex composed of IL-2/IL-15 receptor beta chain and the common gamma chain. IL-15 is secreted by mononuclear phagocytes, and some other cells, following infection by virus(es). This cytokine induces cell proliferation of natural killer cells; cells of the innate immune system whose principal role is to kill virally infected cells. The sequence of IL-15 is shown as SEQ ID NO. 31.

SEQ ID NO. 31
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

The cytokine-based transduction enhancer may comprise one of the following sequences, or a variant thereof:

(membrane-IL7)
SEQ ID NO. 32
MAHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEHSGGGSPAKPTTTPAPRPPTPAPT
IASQPLSLRPEACRPAAGGAVHTRGLDFACDIYIWAPLAGTCGVLLLSLV
ITLYCNHRNRRRVCKCPRPVV
(membrane-IL15)
SEQ ID NO. 33
MGLVRRGARAGPRMPRGWTALCLLSLLPSGFMAGIHVFILGCFSAGLPKT
EANWVNVISDLKKIEDLIQSMHIDATLYTESDVHPSCKVTAMKCFLLELQ
VISLESGDASIHDTVENLIILANNSLSSNGNVTESGCKECEELEEKNIKE
FLQSFVHIVQMFINTSSPAKPTTTPAPRPPTPAPTIASQPLSLRPEACRP
AAGGAVHTRGLDFACDIYIWAPLAGTCGVLLLSLVITLYCNHRNRRRVCK
CPRPVV

The cytokine-based transduction enhancer may comprise a variant of the sequence shown as SEQ ID NO. 32 or 33 having at least 80, 85, 90, 95, 98 or 99% sequence identity, provided that the variant sequence is a cytokine-based transduction enhancer having the required properties i.e. the capacity to activate a T cell when present in the envelope protein of a retroviral or lentiviral vector.

### Illustrative Advantages of Transduction Enhancers

In some embodiments, the present disclosure provides a viral vector with a built-in transduction enhancer. The vector may have the capability to both stimulate the T-cell and to also effect gene insertion. This may produce one or more advantages, including: (1) simplifying the process of T-cell engineering, as only one component needs to be added; (2) avoiding removal of beads and the associated reduction in yield as the virus is labile and does not have to be removed; (3) reducing the cost of T-cell engineering as only one component needs to be manufactured; (4) allowing greater design flexibility, as each T-cell engineering process will involve making a gene-transfer vector, the same product can also be made with a transduction enhancer to “fit” the product; (5) shortening the production process: in soluble antigen/bead-based approaches the mitogen and the vector are typically given sequentially separated by one, two or sometimes three days, this can be avoided with the retroviral vector of the present invention since transduction enhancement and viral entry are synchronized and simultaneous; (6) simplifying engineering as there is no need to test a lot of different fusion proteins for expression and functionality; (7) allowing for the possibility to add more than one signal at the same time; and (8) allowing for the regulation of the expression and/or expression levels of each signal/protein separately.

### Illustrative Embodiments of Viral Vectors Comprising Transduction Enhancers

In some embodiments, the viral envelope comprises one or more transduction enhancers. In some embodiments, the transduction enhancers include T cell activation receptors, NK cell activation receptors, and/or co-stimulatory molecules. In some embodiments, one or more transduction enhancers comprise one or more of anti-CD3scFv, CD86, and CD137L. In some embodiments, the transduction enhancers comprise every one of anti-CD3 scFv, CD86, and CD137L.

In some embodiments, the transduction enhancer comprises a mitogenic stimulus, and/or a cytokine stimulus, which is incorporated into a retroviral or lentiviral capsid, such that the virus both activates and transduces T cells. This removes the need to add vector, mitogen and cytokines separately. In some embodiments, the transduction enhancer comprises a mitogenic transmembrane protein and/or a cytokine-based transmembrane protein that is included in the producer or packaging cell, which get(s) incorporated into the retrovirus when it buds from the producer/packaging cell membrane. In some embodiments, the transduction enhancers are expressed as separate cell surface molecules on the producer cell rather than being part of the viral envelope glycoprotein.

In some embodiments, the present disclosure provides a retroviral or lentiviral vector having a viral envelope which comprises:
(i) a mitogenic transduction enhancer which comprises a mitogenic domain and a transmembrane domain; and/or
(ii) a cytokine-based transduction enhancer which comprises a cytokine domain and a transmembrane domain.

In some embodiments, the transduction enhancers are not part of a viral envelope glycoprotein. In some embodiments, the retroviral or lentiviral vector comprises a separate viral envelope glycoprotein, encoded by an env gene. Since the mitogenic stimulus and/or cytokine stimulus are provided on a molecule which is separate from the viral envelope glycoprotein, integrity of the viral envelope glycoprotein is maintained and there is no negative impact on viral titre.

In some embodiments, there is provided a retroviral or lentiviral vector having a viral envelope which comprises:
(i) a viral envelope glycoprotein: and
(ii) a mitogenic transduction enhancer having the structure: M-S-TM
in which M is a mitogenic domain; S is an optional spacer and TM is a transmembrane domain; and/or
(iii) a cytokine-based transduction enhancer which comprises a cytokine domain and a transmembrane domain.

In some embodiments, the mitogenic transduction enhancer and/or cytokine-based transduction enhancer are not part of the viral envelope glycoprotein. In some embodiments, they exist as separate proteins in the viral envelope and are encoded by separate genes. In some embodiments, the mitogenic transduction enhancer has the structure:
M-S-TM
in which M is a mitogenic domain; S is an optional spacer and TM is a transmembrane domain.

In some embodiments, the mitogenic transduction enhancer binds an activating T-cell surface antigen. In some embodiments, the antigen is CD3, CD28, CD134 or CD137. The mitogenic transduction enhancer may comprise an agonist for such an activating T-cell surface antigen.

The mitogenic transduction enhancer may comprise the binding domain from an antibody such as OKT3, 15E8, TGN1412; or a costimulatory molecule such as OX40L or 41 BBL. The viral vector may comprise two or more mitogenic transduction enhancers in the viral envelope. For example, the viral vector may comprise a first mitogenic transduction enhancer which binds CD3 and a second mitogenic transduction enhancer which binds CD28. The cytokine-based transduction enhnacer may, for example, comprise a cytokine selected from IL2, IL7 and IL15.

In some embodiments, there is provided a retroviral or lentiviral vector having a viral envelope which comprises:
(a) a first mitogenic transduction enhancer which binds CD3; and
(b) a second mitogenic transduction enhancer which binds CD28.

In some embodiments, there is provided a retroviral or lentiviral vector having a viral envelope which comprises:
(a) a first mitogenic transduction enhancer which binds CD3;
(b) a second mitogenic transduction enhancer which binds CD28; and
(c) a cytokine-based transduction enhancer which comprises IL2.

In some embodiments, there is provided a retroviral or lentiviral vector having a viral envelope which comprises:
(a) a first mitogenic transduction enhancer which binds CD3;
(b) a second mitogenic transduction enhancer which binds CD28;
(c) a cytokine-based transduction enhancer which comprises IL7; and
(d) a cytokine-based transduction enhancer which comprises IL15.

### T Cell Activator Proteins

The present disclosure also provides a viral vector comprising a polynucleotide comprising a sequence encoding a T cell activator protein or T cell activator protein complex. As referred to herein, the terms “T cell activator protein” and “T cell activator protein complex” may be used interchangeably and may refer to a single protein or a complex of separate proteins. In some embodiments, the viral vector transduces a host T cell with the polynucleotide encoding the T cell activator protein such that the T cell expresses said protein. The T cell activator protein may then be engaged to activate the transduced T cell. In some embodiments, the T cell activator protein is a drug-inducible T cell activator protein. In some embodiments, the T cell activator protein forms a chemical-induced signaling complex. In some embodiments, the T cell activator protein forms an engineered complex that initiates a signal into the interior of a cell as a direct outcome of ligand-induced dimerization. The T cell activator protein may be comprised in a homodimer (dimerization of two identical components) or a heterodimer (dimerization of two distinct components). The T cell activator protein complex may be a synthetic complex as described herein. One of skill in the art will recognize that the component parts of the T cell activator protein complex may be composed of a natural or a synthetic component useful for incorporation into the complex. Thus, the examples provided herein are not intended to be limiting. Additional T cell activator proteins that may be implemented herein may be found in WO 2016/139463 and WO 2018/111834, the disclosures of which are incorporated in their entireties herein.

In some embodiments, the T cell activator protein sequence can have a first and a second sequence. The first sequence may encode a first T cell activator protein complex component that can comprise a first extracellular binding domain or portion thereof, a hinge domain, a transmembrane domain, and a signaling domain or portion thereof. The second sequence encodes a second T cell activator protein complex component that can comprise a second extracellular binding domain or a portion thereof, a hinge domain, a transmembrane domain, and a signaling domain or portions thereof. In some embodiments, the first and second components may be positioned such that when expressed, they dimerize in the presence of a ligand.

As used herein, the terms “rapamycin activated cytokine receptor” or “RACR” refer interchangeably to a multipartite receptor that inducibly generates an intracellular signal that promotes proliferation and/or activity of a cell in the presence of rapamycin. The RACR may transduce an IL2-like signal in a T cell in the presence of rapamycin through IL-2R intracellular domain(s) or variants thereof.

In some embodiments, the disclosure provides a protein sequence or sequences for heterodimeric two component T cell activator protein complex. In some embodiments, the first component is an IL2Rγ complex. In some embodiments, the IL2Rγ complex comprises an amino acid sequence as set forth in SEQ ID NO: 34

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 34).

In some embodiments, the IL2Rγ complex comprises an amino acid sequence as set forth in SEQ ID NO: 36.

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 36).

In some embodiments, the IL2Rγ complex comprises an amino acid sequence as set forth in SEQ ID NO: 37.

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 37).

In some embodiments, the IL2Rγ complex comprises an amino acid sequence as set forth in SEQ ID NO: 38.

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 38)

In some embodiments, the protein sequence for the first T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first T cell activator protein complex component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 1, 3, 5, or 7, or has a sequence identity that is within a range defined by any two of the aforementioned percentages.

In some embodiments, the second T cell activator protein complex component is an IL2Rβ complex. In some embodiments, the IL2Rβ complex comprises an amino acid sequence as set forth in SEQ ID NO: 39.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQ
GEFRALNARLPLNTDAYLSLQELQGQDPTHLV; SEQ ID NO: 39).

In some embodiments, the IL2Rβ complex comprises an amino acid sequence as set forth in SEQ ID NO: 40.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 40).

In some embodiments, the IL2Rβ complex comprises an amino acid sequence as set forth in SEQ ID NO: 41.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 41).

In some embodiments, the IL2Rβ complex comprises an amino acid sequence as set forth in SEQ ID NO: 42.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 42).

In some embodiments, the second T cell activator protein complex component is an IL7Rα complex. In some embodiments, the IL7Rα complex comprises an amino acid sequence as set forth in SEQ ID NO: 43.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 43).

In some embodiments, the protein sequence for the second T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second T cell activator protein complex component. In some embodiments, the protein sequence of the second T cell activator protein complex component, comprising the second extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 39-43, or has a sequence identity that is within a range defined by any two of the aforementioned percentages.

In some embodiments, the protein sequence may include a linker. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 44 (GGGS; SEQ ID NO: 44), SEQ ID NO: 45 (GGGSGGG; SEQ ID NO: 45) or SEQ ID NO: 46 (GGG; SEQ ID NO: 46). Embodiments also comprise a nucleic acid sequence encoding SEQ ID NOs: 44-46. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

In some embodiments is provided a protein sequence or sequences for homodimeric two component T cell activator protein complex. In some embodiments, the first T cell activator protein complex component is an IL2Rγ complex. In some embodiments, the IL2Rγ complex comprises an amino acid sequence as set forth in SEQ ID NO: 47.

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 47).

In some embodiments, the protein sequence for the first T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first T cell activator protein complex component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 47 or has a sequence identity that is within a range defined by any two of the aforementioned percentages.

In some embodiments, the second T cell activator protein complex component is an IL2Rβ complex or an IL2Rα complex. In some embodiments, the IL2Rβ complex comprises an amino acid sequence as set forth in SEQ ID NO: 48.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 48).

In some embodiments, the IL2Rα complex comprises an amino acid sequence as set forth in SEQ ID NO: 49.

(MALPVTALLLPLALLLHAARPILWHEMWHEGLEEASRLYFGERNVKGMF
EVLEPLHAMMERGPQTLKETSFNQAYGRDLMEAQEWCRKYMKSGNVKDLL
QAWDLYYHVFRRISKGKDTIPWLGHLLVGLSGAFGFIILVYLLINCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV; SEQ ID NO: 49).

In some embodiments, the protein sequence for the second T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second T cell activator protein complex component. In some embodiments, the protein sequence of the second T cell activator protein complex component, comprising the second extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NO: 48 or SEQ ID NO: 49, or has a sequence identity that is within a range defined by any two of the aforementioned percentages.

In some embodiments, the sequences for the homodimerizing two component T cell activator protein complex incorporate FKBP F36V domain for homodimerization with the ligand AP1903.

In some embodiments is provided a protein sequence or sequences for single component homodimerization T cell activator protein complex. In some embodiments, the single component T cell activator protein complex is an IL7Rα complex. In some embodiments, the IL7Rα complex comprises an amino acid sequence as set forth in SEQ ID NO: 50.

(MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISPD
YAYGATGHPGIIPPHATLVFDVELLKLGEGSNTSKENPFLFALEAVVISV
GSMGLIISLLCVYFWLERTMPRIPTLKNLEDLVTEYHGNFSAWSGVSKGL
AESLQPDYSERLCLVSEIPPKGGALGEGPGASPCNQHSPYWAPPCYTLKP
ET; SEQ ID NO: 50).

In some embodiments, the at least one T-cell activator protein comprises a first receptor protein comprising a first dimerization domain and a second receptor protein comprising a second dimerization domain, wherein the first dimerization domain and the second dimerization domain specifically bind to one another in response to a molecule. The molecule bound by the T cell activator protein, alternatively termed the term “ligand” or “agent”, refers to a molecule that has a desired biological effect. In some embodiments, a ligand is recognized by and bound by an extracellular binding domain, forming a tripartite complex comprising the ligand and two binding T cell activator protein complex components. Ligands include, but are not limited to, proteinaceous molecules, comprising, but not limited to, peptides, polypeptides, proteins, post-translationally modified proteins, antibodies etc.; small molecules (less than 1000 daltons), inorganic or organic compounds; and nucleic acid molecules comprising, but not limited to, double-stranded or single-stranded DNA, or double-stranded or single-stranded RNA (e.g., antisense, RNAi, etc.), aptamers, as well as triple helix nucleic acid molecules. Ligands can be derived or obtained from any known organism (comprising, but not limited to, animals (e.g., mammals (human and non-human mammals)), plants, bacteria, fungi, and protista, or viruses) or from a library of synthetic molecules. In some embodiments, the ligand is a protein, an antibody, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, or AP1903, or metabolites, derivatives, and/or combinations thereof. In some embodiments, the ligand is an IMID-class drug (e.g. thalidomide, pomalidimide, lenalidomide or related analogues).

In some embodiments, the molecule is selected from FK1012, tacrolimus (FK506), FKCsA, rapamycin, coumermycin, gibberellin, HaXS, TMP-HTag, and ABT-737 or functional derivatives thereof.

### Adaptor Molecules

As used herein, the term “adaptor molecule” refers to any molecule having a hapten moiety recognizable by a receptor linked to a targeting moiety. A “targeting moiety” is any molecule that specifically or non-specifically binds to a target cell. In some embodiments, the targeting moiety is a protein. Illustrative proteins for use as targeting moieties include those described in U.S. Pat. No. 9,233,125, the disclosure of which is incorporated by reference herein in its entirety. In some embodiments, the protein is an antibody or an antigen-binding fragment thereof. In some embodiments, the antibody or an antigen-binding fragment thereof is specific for a tumor antigen. Illustrative proteins include, but are not limited to, anti-cancer-based monoclonal antibodies such as cetuximab (anti-EGFR), nimotuzumab (anti-EGFR), panitumumab (anti-EGFR), rituximab (anti-CD20), omalizumab (anti-CD20), tositumomab (anti-CD20), trastuzumab (anti-Her2), gemtuzumab (anti-CD33), alemtuzumab (anti-CD52), and bevacizumab (anti-VEGF).

In some embodiments, the targeting moiety is a small molecule. Illustrative proteins for use as targeting moieties include those described in U.S. patent application Ser. Nos. 15/296,666, 16/092,054, and 16/253,562, each of which is incorporated by reference herein in its entirety. Illustrative small molecules useful as targeting moeities include, without limitation, a folate, dicarboxypropyl)ureido] pentanedioic acid (DUPA), an NK-1R ligand, a CAIX ligand, a ligand of gamma glutamyl transpeptidase, an NKG2D ligand, or a cholecystokinin 2 receptor (CCK2R) ligand.

In some embodiments the targeting moiety is a lipid, or more particularly a phospho-lipid ether (PLE). Illustrative proteins for use as targeting moieties include those described in International Pat. Appl. Pub. Nos. WO2018148224, WO2019060425, and WO2018148224, each of which is incorporated by reference herein in its entirety. In some embodiments, the lipid comprises a polar head group and a hydrophobic group. In some embodiments, the hydrophobic group is a carbon chain or a fatty acid such as an aliphatic chain. In some embodiments, the carbon chain or fatty acid is saturated or unsaturated. In some embodiments, the hydrophobic group comprises an alkyl, alkenyl or alkynyl group. In some embodiments, the hydrophobic group comprises a terpenoid lipid such as a steroid or cholesterol or it comprises an aromatic ring. In some embodiments, the hydrophobic group comprises an ether linkage, wherein the ether linkage is between the polar head group and the aliphatic chain. In some embodiments, the lipid is a phospholipid ether. In some embodiments, the polar head comprises a choline, a phosphatidylcholine, sphingomyelin, phosphoethanolamine group, an oligosaccharide residue, a sugar residue, phosphatidyl serine or phosphatidyl inositol. In some embodiments, the sugar is a glycerol. In some embodiments, the target moiety is a hapten, poly(his) tag, Strep-tag, FLAG-tag, VS-tag, Myc-tag, HA-tag, NE-tag, biotin, digoxigenin, dinitrophenol or fluorescein. In some embodiments, the hydrophobic group comprises a carbon alkyl chain, wherein the carbon alkyl chain comprises at least 8, 9, 10, 11, 12, 13, 14, 15, 16, 17, 18, 19, 20, 21 or 22 carbons or any number that is within a range defined by any two aforementioned values. In some embodiments, the carbon alkyl chain comprises 8-22 carbons, such as 8-12, 12-14, 14-16, or 16-22 carbons. In some embodiments, the polar-head group comprises phosphocholine, a piperidine moiety or a trimethylarseno-ethyl-phosphate moiety. In some embodiments, the lipid further comprises a spacer that separates the target moiety from the polar head group. In some embodiments, the spacer comprises a PEG spacer, a Hapten (2×) spacer, a Hapten (3×) spacer, a Hapten (4×) spacer, a Hapten (5×) spacer, or an alkane chain. In some embodiments, the spacer comprises poly(carboxybetaine), peptides, polyglycidols, polyethylene, Polyanhydrides, Polyphosphoesters, Polycaprolactone or Poly(ethylene oxide). In some embodiments, the PEG spacer comprises 1, 2, 3, 4, 5, 6, 7, 8, 9, 10, 11, 12, 13, 14, 15, 16, 17, 18, 19, 20 or 21 PEG molecules, or any amount of PEG molecules that is within a range defined by any two aforementioned values. In some embodiments, the lipid is intercalated in a lipid bilayer of a target cell, such as a cancer cell.

In some embodiments, the targeting moiety is an antibody or an antigen-binding fragment thereof. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The term is used in the broadest sense and includes polyclonal and monoclonal antibodies, including intact antibodies and functional (antigen-binding) antibody fragments, including fragment antigen-binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The term “antibody fragment” refers to a portion of an intact antibody and refers to the antigenic determining variable regions of an intact antibody. Examples of antibody fragments include, but are not limited to, fragment antigen-binding (Fab) fragments, F(ab′)2 fragments, Fab′ fragments, Fv fragments, recombinant IgG (rlgG) fragments, single chain antibody fragments, including single chain variable fragments (scFv), single domain antibodies (e.g., sdAb, sdFv, nanobody) fragments, diabodies, and multispecific antibodies formed from antibody fragments. In a specific embodiment, the antibody fragment is an scFv. Non-limiting examples of an antibody or binding fragment thereof include monoclonal antibodies, bispecific antibodies, Fab, Fab2, Fab3, scFv, Bis-scFv, minibody, triabody, diabody, tetrabody, VhH domain, V-NAR domain, IgNAR, and camel Ig. Additional examples of an antibody are IgG (e.g., IgG1, IgG2, IgG3, or IgG4), IgM, IgE, IgD, and IgA. Non-limiting examples of antibodies include human antibodies, humanized antibodies, or chimeric antibodies. Non-limiting examples of recombinant antibodies include antibodies that specifically bind to a tumor antigen.

In some embodiments, the targeting moiety comprises a phospholipid ether (PLE). In some embodiments, the targeting moiety comprises a folate. In some embodiments, the adaptor molecule comprises HPF conjugated to a PLE. In some embodiments, the adaptor molecule comprises a HPF-FITC-PEG3-C18-alkylphospholipid. In some embodiments, the adaptor molecule comprises a HPF-{linker}-erufosine.

As used herein, a “hapten” refers to any moiety capable of being specifically recognized by a receptor, such as a chimeric antigen receptor or the like. In some embodiments, the adaptor molecule contains more than one hapten, such as 2, 3, 4, 5, 6, or more haptens. The haptens may be the same or different from one another. In general the hapten (or haptens) is covalently linked to the targeting moiety directly or via a spacer. Several types of “spacers” are contemplated for use with embodiments described herein including, without limitation, a poly(carboxybetaine), peptide, polyglycidols, polyethylene, polyanhydrides, polyphosphoesters, polycaprolactone, poly(ethylene oxide), PEG spacer, a small peptide or an alkane chain. In some embodiments, the alkane spacer can comprise 2, 3, 4, 5, 6, 7, 8, 9, 10, 11, 12, 13, 14, 15, 16, 17, or 18 carbons, or any number of carbons in between a range defined by any two aforementioned values. In some embodiments, the PEG spacer comprises 1, 2, 3, 4, 5, 6, 7, 8, 9, 10, 11, 12, 13, 14, 15, 16, 17, 18, 19, 20 or 21 PEG molecules, or any amount of PEG molecules that is within a range defined by any two aforementioned values.

Various small-molecules haptens are known in the art. Illustrative haptens for use in the compositions and methods of the disclosure include those described in International Pat. Appl. Pub. No. WO2018148224, which is incorporated by reference herein in its entirety. In another aspect, the present disclosure contemplates selecting a novel hapten and generating an antibody specific to that hapten using anti-hapten antibody production techniques known in the art. In some embodiments, the hapten comprises a fluorescein. Recombinant human antibody E2 is an antibody capable of binding to fluorescein. In some embodiments, the hapten comprises a fluorescein and the hapten-binding receptor comprises an anti-fluorescein antibody or antigen-binding fragment thereof, e.g., antibody E2. In some embodiments, the hapten comprises 2,4-dinitrophenol (DNP).

In some embodiments, the adaptor molecule comprises one or more masking moieties covalently linked to the hapten, thereby producing a “masked hapten” comprising at least one hapten and at least one masking moiety. A “masking moiety” is a chemical moiety that prevents or inhibits binding to the masked form of the hapten of ligands or receptors that are normally able to bind the unmasked form of the hapten. A masking moiety may include a protective group to prevent recognition of the hapten by blocking binding and recognition of a hapten-binding receptor (e.g., a chimeric antigen receptor) that is specific for the hapten. When the adaptor molecule is integrated into a cell, wherein the cell exists in a tumor environment or site of reactive oxygen species, the masking moiety can be self-cleaved, thus allowing binding and recognition of the hapten by the chimeric antigen receptor. In some embodiments, the targeting moiety is a lipid that is a phospholipid ether. In some embodiments, the masking moiety comprises a phenolic hydroxyl group or PEG. In some embodiments, the phenolic hydroxyl group is bound to a hydroxyl on a xanthene moiety of fluorescein. In some embodiments, the masking moiety is bound to the adaptor molecule by a cleavable moiety, which is optionally configured to be specifically cleavable in a tumor microenvironment. In some embodiments, the cleavable moiety, which is configured to be cleavable in a tumor microenvironment, is cleaved by a reactive oxygen species reaction, an acidic pH, hypoxia, or nitrosylation. In some embodiments, the phospholipid ether comprises a hapten and the CAR is joined to said phospholipid ether through an interaction with said hapten. In some embodiments, the phospholipid ether comprises a polar-head group and a carbon alkyl chain. In some embodiments, the carbon alkyl chain comprises at least 8, 9, 10, 11, 12, 13, 14, 15, 16, 17, 18, 19, 20, 21 or 22 carbons or any number that is within a range defined by any two aforementioned values. In some embodiments, the carbon alkyl chain comprises 8-22 carbons, such as 8-12, 12-14, 14-16, or 16-22 carbons. In some embodiments, the masking moiety is removed when the composition is within an acidic environment. In some embodiments, the acidic environment comprises a pH or 4, 5, 6 or 6.5 or any pH in between a range defined by any two aforementioned values. In some embodiments, the masking moiety is removed by nitrosylation.

In some embodiments, the masked hapten is configured to permit a chemical reaction to remove the masking moiety from the hapten. In some embodiments, the masked hapten is configured to permit reactive oxygen species to remove the masking moiety from the hapten. In some embodiments, the masked hapten comprises a hydroxyphenyl group. In some embodiments, the masking moiety comprises a 2,4-dinitrophenol (DNP) group. In some embodiments, the hapten comprises a fluorescein. In some embodiments, the masked hapten comprises a hydroxyphenyl fluorescein (HPF). In some embodiments, the masked hapten comprises a fluorescein-DNP.

### Hapten-Binding Receptors

The present disclosure also provides receptors that bind to the adaptor molecules, particularly that bind to the hapten comprised by the adaptor molecules. The hapten and the adaptor molecule may be any of those set forth in the foregoing sections. In some embodiments, the hapten-binding receptor is a cell surface receptor that naturally binds to the hapten. In some embodiments, the hapten-binding receptor is partially or wholly synthetic. In some embodiments, the hapten-binding receptor is a recombinant protein. In some embodiments, the hapten-binding receptor is a chimeric receptor.

In some embodiments, the hapten-binding receptor is a chimeric antigen receptor. The terms “Chimeric antigen receptor” or “CAR” or “Chimeric T cell receptor” refer to a synthetically designed receptor comprising a ligand binding domain of an antibody or other protein sequence that binds to a molecule, a transmembrane domain, one or more intracellular signaling domains, and one or more co-stimulatory domains. The ligand binding domain is linked via a spacer domain to one or more intracellular signaling domains of a T cell or other receptors, such as a costimulatory domain. Chimeric receptors can also be referred to as artificial T cell receptors, chimeric T cell receptors, chimeric immunoreceptors, and chimeric antigen receptors (CARs). These CARs are engineered receptors that can graft an arbitrary specificity onto an immune receptor cell. In some embodiments, the spacer for the chimeric antigen receptor is selected (e.g., for a particular length of amino acids in the spacer) to achieve desired binding characteristics for the CAR. CARs having varying lengths of spacers, e.g., presented on cells are then screened for the ability to bind or interact with an adaptor molecule and/or hapten to which the CAR is directed.

In some embodiments herein, the CAR comprises one or more intracellular signaling domains. In some embodiments, the intracellular signaling domain is derived from CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83, or a portion thereof.

In some embodiments, the CAR comprises one or more co-stimulatory domains. A “co-stimulatory domain” refers to a signaling moiety that provides to T cells a signal which, in addition to the primary signal provided by for instance the CD3 zeta chain of the TCR/CD3 complex, mediates a T cell response, including, but not limited to, activation, proliferation, differentiation, cytokine secretion, and the like. A co-stimulatory domain can include all or a portion of, but is not limited to, CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83. In some embodiments, the co-stimulatory domain is an intracellular signaling domain that interacts with other intracellular mediators to mediate a cell response including activation, proliferation, differentiation and cytokine secretion, and the like. In some embodiments, herein the co-stimulatory domain comprises 41bb and CD3zeta. In some embodiments, a T cell is provided, wherein the T cell comprises a CAR specific for the hapten on the adaptor molecule. In some embodiments, the T cell further comprise an 806 CAR (anti-EGFR(806)(41BB-CD3zeta CAR).

In some embodiments, said chimeric receptor has at least 80% amino acid identity,

at least 90% amino acid identity or at least 95% amino acid identity to:

(SEQ ID NO: 53)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLGSTSGSGKPGSGEGSTKGQVQLVESGGNLVQPGGSLRLSCA
ASGFTFGSFSMSWVRQAPGGGLEWVAGLSARSSLTHYADSVKGRFTISRD
NAKNSVYLQMNSLRVEDTAVYYCARRSYDSSGYWGHFYSYMDVWGQGTLV
TVSSESKYGPPCPPCPMFWVLVVVGGVLACYSLLVTVAFIIFWVKRGRKK
LLYIFKQPFMRPVQTTQEEDGCSCRFPEEEEGGCELRVKFSRSADAPAYQ
QGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR,

or a variant thereof sharing the same complementarity determining regions.

In some embodiments, the chimeric antigen receptor comprises an anti-FITC scFv conjugated to a CD28 transmembrane span and a 4-1BB zeta signaling tail via a short IgG4 linker.

Illustrative chimeric antigen receptors comprising an anti-FITC scFv include those described in U.S. Patent Appl. No. 63/052,806, which is incorporated by reference herein in its entirety.

In some embodiments, the hapten-binding receptor comprises a hapten-specific antigen-binding fragment of an antibody. In some embodiments, the antigen-binding fragment comprises an Fab fragment or a single-chain Fv fragment (scFv). In some embodiments, the receptor that specifically binds to the hapten comprises a hapten-specific chimeric antigen receptor.

In some embodiments, the hapten-binding receptor is a T cell receptor (TCR) or a functional portion thereof. A “T cell receptor” or “TCR” refers to a molecule that is found on the surface of T lymphocytes or T cells that is responsible for the recognition of fragments of antigen bound to a major histocompatibility complex molecule.

In some embodiments, the hapten-binding receptor is a dimerization activated receptor initiation complex (DARIC). A DARIC provides a binding component and a signaling component that are each expressed as separate fusion proteins but contain an extracellular multimerization mechanism (bridging factor) for recoupling of the two functional components on a cell surface (see U.S. Pat. Appl. No. 2016/0311901, hereby expressly incorporated by reference in its entirety). Importantly, the bridging factor in the DARIC system forms a heterodimeric receptor complex, which does not produce significant signaling on its own. The described DARIC complexes only initiate physiologically relevant signals following further co-localization with other DARIC complexes. Thus, they do not allow for the selective expansion of desired cell types without a mechanism for further multimerization of DARIC complexes (such as by e.g., contact with a tumor cell that expresses a ligand bound by a binding domain incorporated into one of the DARIC components). Thus, as used herein, in some embodiments, the binding domains incorporated into the DARIC components bind to a hapten comprised by an adaptor molecule as disclosed herein.

In some embodiments, the hapten-binding portion of a hapten-binding receptor may comprise an antigen-binding portion of an antibody or an antigen-binding antibody derivative. An antigen-binding portion or derivative of an antibody may be a Fab, Fab′, F(ab′)2, Fd, Fv, scFv, a diabody, a linear antibody, a single-chain antibody, a minibody, or the like. In some embodiments, the hapten-binding portion of a hapten-binding receptor may comprise a DARPin or centyrin.

The hapten-binding receptor may bind to a molecule associated with a disease or disorder. As used herein, the molecule may be a hapten comprised by an adaptor molecule. In some embodiments, the hapten to which the hapten-binding receptors bind or interact can be presented on a substrate, such as a membrane, bead, or support (e.g., a well) or a binding agent, such as a lipid (e.g., PLE), hapten, ligand, or antibody, or binding fragment thereof. In some embodiments, the adaptor molecule is a binding agent that has specificity for an antigen present on a cancer cell. In some embodiments, the adaptor molecule is a binding agent that has specificity for a pathogen, such as a virus or bacterium. By one approach, the substrate or adaptor molecule comprising the desired hapten is contacted with a plurality of cells comprising a hapten-binding receptor specific for said hapten and the level or amount of binding of the cells comprising the hapten-binding receptor to the hapten present on the substrate or binding agent is determined. Such an evaluation of binding may include staining for cells bound to adaptor molecules or evaluation of fluorescence or loss of fluorescence. Again, modifications to the hapten-binding receptor structure, such as varying spacer lengths, can be evaluated in this manner. In some approaches, a target cell is also provided such that the method comprises contacting a cell, such as a T cell, which comprises a hapten-binding receptor that is specific for an adaptor molecule comprising a target moiety and a hapten, in the presence of a target cell, such as a cancer cell or bacterial cell, or a target virus and evaluating the binding of the cell comprising the hapten-binding receptor to the adaptor molecule and/or evaluating the binding of the cell comprising the hapten-binding receptor to the target cell or target virus. The variation of the different elements of the hapten-binding receptor can, for example, lead to stronger binding affinity for a specific epitope or antigen.

In some embodiments described herein, the hapten-binding receptor is specific for a lipid or peptide that targets a tumor or cancer cell, wherein the lipid or peptide comprises a hapten and the hapten-binding receptor can specifically bind to said lipid through an interaction with said hapten. In some embodiments, the lipid is a phospholipid ether. In some embodiments described herein, the hapten-binding receptor is specific for a phospholipid ether, wherein the phospholipid ether comprises a hapten and the hapten-binding receptor specifically binds to said phospholipid ether through an interaction with said hapten.

In some embodiments, the hapten-binding receptor is specific for a hapten affixed to an antibody or binding fragment thereof, wherein the hapten-binding receptor specifically binds to said antibody or binding fragment thereof through an interaction with said hapten. Exemplary haptens which can be conjugated to said antibody or binding fragment thereof include a poly(his) tag, Strep-tag, FLAG-tag, VS-tag, Myc-tag, HA-tag, NE-tag, biotin, digoxigenin, dinitrophenol, green fluorescent protein (GFP), yellow fluorescent protein, orange fluorescent protein, red fluorescent protein, far red fluorescent protein, or fluorescein (e.g., fluorescein isothiocyanate (FITC)). In some embodiments, the antibody or binding fragment thereof is specific for an antigen or ligand present on a cancer cell or a pathogen (e.g., viral or bacterial pathogen). In some embodiments, the antibody or binding fragment thereof is specific for an antigen or ligand present on a tumor cell, a virus, preferably a chronic virus (e.g., a hepatitis virus, such as HBV or HCV, or HIV), or a bacterial cell.

In some embodiments, the hapten-binding receptor nucleic acid comprises a polynucleotide coding for a transmembrane domain. The transmembrane domain provides for anchoring of the chimeric receptor in the membrane.

In some embodiments, a complex is provided, wherein the complex comprises a hapten-binding receptor joined to a lipid wherein the lipid comprises a hapten and the hapten-binding receptor is joined to said lipid through an interaction with said hapten.

In some embodiments, a complex is provided, wherein the complex comprises a hapten-binding receptor joined to an antibody or binding fragment thereof, wherein the antibody or binding fragment thereof comprises a hapten (e.g., a poly(his) tag, Strep-tag, FLAG-tag, VS-tag, Myc-tag, HA-tag, NE-tag, biotin, digoxigenin, dinitrophenol, green fluorescent protein (GFP), yellow fluorescent protein, orange fluorescent protein, red fluorescent protein, far red fluorescent protein, or fluorescein (e.g., fluorescein isothiocyanate (FITC)) and the hapten-binding receptor is joined to said antibody or binding fragment thereof through an interaction with said hapten. In some embodiments, the antibody or binding fragment thereof is further joined to an antigen or ligand present on a cancer cell or a pathogen (e.g., viral or bacterial pathogen). In some embodiments, the antibody or binding fragment thereof is joined to an antigen or ligand present on a tumor cell, a virus, preferably a chronic virus (e.g., a hepatitis virus, such as HBV or HCV, or HIV), or a bacterial cell. In some embodiments, the hapten is present on an antibody or binding fragment thereof, which are specific for an antigen on a cancer cell or pathogen (e.g., a virus or bacterial cell), and said hapten is bound by a hapten-binding receptor present on the surface of a cell (e.g., a T cell) such that the cell having the hapten-binding receptor is redirected to the cancer cell or pathogen.

In some embodiments, the hapten-binding receptor or T cell activator protein of the present disclosure confers resistance to an immunosuppressive or anti-proliferative agent to the immune cell. In some cases, the lentiviral vector facilitates selective expansion of target cells by conferring resistance to an immunosuppressive or anti-proliferative agent to transduced cells, facilitating selective expansion of target cells. The present disclosure provides lentiviral vectors that comprise any of the nucleic sequences that confer resistance to an immunosuppressive or anti-proliferative agent. Examples of immunosuppressive or anti-proliferative agents include, without limitation, rapamycin or a derivative thereof, a rapalog or a derivative thereof, tacrolimus or a derivative thereof, cyclosporine or a derivative thereof, methotrexate or derivatives thereof, and mycophenolate mofetil (MMF) or derivatives thereof. Various resistance genes are known in the art. Resistance to rapamycin may be conferred by a polynucleotide sequence encoding the protein domain FRb, found in the mTOR domain and known to be the target of the FKBP-rapamycin complex. Resistance to tacrolimus may be conferred by a polynucleotide sequence encoding the calcineurin mutant CNa22 or calcineurin mutant CNb30. Resistance to cyclosporine may be conferred by a polynucleotide sequence encoding the calcineurin mutant CNa12 or calcineurin mutant CNb30. These calcineurin mutants are described in Brewin et al. (2009) Blood 114:4792-803. Resistance to methotrexate can be provided by various mutant forms of di-hydrofolate reducatse (DHFR), Volpato et al. (2011) J Mol Recognition 24:188-198, and resistance to MMF can be provided by various mutant forms of inosine monophosphate dehydrogenase (IMPDH), Yam et al. (2006) Mol Ther 14:236-244.

Immunosuppressive or anti-proliferative agents (e.g., immunosuppressive drugs) are commonly used prior to, during, and/or after ACT. In some cases, use of an immunosuppressive drug may improve treatment outcomes. In some cases, use of an immunosuppressive drug may diminish side effects of treatment, such as, without limitation, acute graft-versus-host disease, chronic graft-versus-host disease, and post-transplant lymphoproliferative disease. The present disclosure contemplates use of immunosuppressive drugs with any of the methods of treating or preventing a disease or condition of the present disclosure, including, without limitation, methods of the present disclosure in which the lentiviral vector confers resistance to an immunosuppressive drug to transduced cells.

### Polynucleotides

The present disclosure also relates to nucleic acids and polynucleotides encoding the disclosed transduction enhancers, T cell activator proteins, adaptor molecules, and hapten-binding receptors. The nucleic acid may be in the form of a construct comprising a plurality of sequences encoding any of the aforementioned proteins. As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence. The nucleic acid may produce a polypeptide which comprises one or more sequences encoding a mitogenic transduction enhancer and/or one or more sequences encoding a cytokine-based transduction enhancer. The cleavage site may be self-cleaving, such that when the polypeptide is produced, it is immediately cleaved into the receptor component and the signalling component without the need for any external cleavage activity.

Various self-cleaving sites are known, including the Foot-and-Mouth disease virus (FMDV) 2a self-cleaving peptide and various variants and 2A-like peptides. The peptide may have the sequence shown as SEQ ID NO. 51 or 52.

SEQ ID NO: 51: RAEGRGSLLTCGDVEENPGP.
SEQ ID NO: 52: QCTNYALLKLAGDVESNPGP.

The co-expressing sequence may be an internal ribosome entry sequence (IBES). The co-expressing sequence may be an internal promoter.

In some embodiments, the polynucleotide encodes a protein that confers resistance to an antiangiogenic agent to the immune cell transduced with it.

### Viral Particle Tagging Proteins

The viral envelope of the viral vector may also comprise a tagging protein which comprises a binding domain which binds to a capture moiety and a transmembrane domain.

The tagging protein may comprise: a binding domain which binds to a capture moiety; a spacer; and a transmembrane domain.

The tagging protein facilitates purification of the viral vector from cellular supernatant via binding of the tagging protein to the capture moiety. ‘Binding domain’ refers to an entity, for example an epitope, which is capable recognising and specifically binding to a target entity, for example a capture moiety. The binding domain may comprise one or more epitopes which are capable of specifically binding to a capture moiety. For example the binding domains may comprise at least one, two, three, four or five epitopes capable of specifically binding to a capture moiety. Where the binding domain comprises more than one epitope, each epitope may be separated by a linker sequence, as described herein.

The binding domain may be releasable from the capture moiety upon the addition of an entity which has a higher binding affinity for the capture moiety compared to the binding domain.

The binding domain may comprise one or more streptavidin-binding epitope(s). For example, the binding domain may comprise at least one, two, three, four or five streptavidin-binding epitopes.

Streptavidin is a 52.8 kDa protein purified from the bacterium Streptomyces avidinii. Streptavidin homo-tetramers have a very high affinity for biotin (vitamin B7 or vitamin H), with a dissociation constant (Kd) ˜10″15 M. Streptavidin is well known in the art and is used extensively in molecular biology and bionanotechnology due to the streptavidin-biotin complex's resistance to organic solvents, denaturants, proteolytic enzymes, and extremes of temperature and pH. The strong streptavidin-biotin bond can be used to attach various biomolecules to one another or on to a solid support. Harsh conditions are needed to break the streptavidin-biotin interaction, however, which may denature a protein of interest being purified.

The binding domain may be, for example, a biotin mimic. A ‘biotin mimic’ may refer to an short peptide sequence (for example 6 to 20, 6 to 18, 8 to 18 or 8 to 15 amino acids) which specifically binds to streptavidin. As described above, the affinity of the biotin/streptavidin interaction is very high. It is therefore an advantage of the present invention that the binding domain may comprise a biotin mimic which has a lower affinity for streptavidin compared to biotin itself.

In particular, the biotin mimic may bind streptavidin with a lower binding affinity than biotin, so that biotin may be used to elute streptavidin-captured retroviral vectors. For example, the biotin mimic may bind streptavidin with a Kd of 1 nM to 100 uM.

The biotin mimic may be selected from the following group: Streptagll, Flankedccstreptag and ccstreptag. The binding domain may comprise more than one biotin mimic. For example the binding domain may comprise at least one, two, three, four or five biotin mimics. Where the binding domain comprises more than one biotin mimic, each mimic may be the same or a different mimic.

The present disclosure also provides viral particles that may be purified and methods of purification of the same. In some embodiments, the viral envelope of the viral vector may also comprise a tagging protein which comprises: a binding domain which binds to a capture moiety; a spacer; and a transmembrane domain, which tagging protein facilitates purification of the viral vector from cellular supernatant via binding of the tagging protein to the capture moiety.

The binding domain of the tagging protein may comprise one or more streptavidin-binding epitope(s). The streptavidin-binding epitope(s) may be a biotin mimic, such as a biotin mimic which binds streptavidin with a lower affinity than biotin, so that biotin may be used to elute streptavidin-captured retroviral vectors produced by the packaging cell. Examples of suitable biotin mimics include: Streptagll, Flankedccstretag, and ccstreptag. The viral vector of the first aspect of the invention may comprise a nucleic acid sequence encoding a T-cell receptor or a chimeric antigen receptor. The viral vector may be a virus-like particle (VLP).

### Production/Packaging Cell Lines

The present disclosure provides a host cell for the production of viral particles according to the disclosure. In some embodiments, the host cell expresses a mitogenic transduction enhancer and/or a cytokine-based transduction enhancer at the cell surface. The host cell may be for the production of viral vectors according to the foregoing embodiments. In some embodiments, the host cell may comprise tagging proteins useful for the purification of the viral particles.

The host cell may be a packaging cell and comprise one or more of the following genes: gag, pol, env and rev. A packaging cell for a retroviral vector may comprise gag, pol and env genes. A packaging cell for a lentiviral vector may comprises gag, pol, env and rev genes.

The host cell may be a producer cell and comprise gag, pol, env and optionally rev genes and a retroviral or lentiviral vector genome. In a typical recombinant retroviral or lentiviral vector for use in gene therapy, at least part of one or more of the gag-pol and env protein coding regions may be removed from the virus and provided by the packaging cell. This makes the viral vector replication-defective as the virus is capable of integrating its genome into a host genome but the modified viral genome is unable to propagate itself due to a lack of structural proteins.

Packaging cells are used to propagate and isolate quantities of viral vectors i.e to prepare suitable titres of the retroviral vector for transduction of a target cell.

In some instances, propagation and isolation may entail isolation of the retroviral gagpol and env (and in the case of lentivirus, rev) genes and their separate introduction into a host cell to produce a packaging cell line. The packaging cell line produces the proteins required for packaging retroviral DNA but it cannot bring about encapsidation due to the lack of a psi region. However, when a recombinant vector carrying a psi region is introduced into the packaging cell line, the helper proteins can package the psi-positive recombinant vector to produce the recombinant virus stock.

A summary of the available packaging lines is presented in “Retroviruses” (1997 Cold Spring Harbour Laboratory Press Eds: J M Coffin, S M Hughes, H E Varmus pp 449).

Packaging cells have also been developed in which the gag, pol and env (and, in the case of lentiviral vectors, rev) viral coding regions are carried on separate expression plasmids that are independently transfected into a packaging cell line, so that three recombinant events are required for wild type viral production.

Transient transfection avoids the longer time required to generate stable vector-producing cell lines and is used if the vector or retroviral packaging components are toxic to cells. Components typically used to generate retroviral/lentivial vectors include a plasmid encoding the Gag/Pol proteins, a plasmid encoding the Env protein (and, in the case of lentiviral vectors, the rev protein), and the retroviral/lentiviral vector genome. Vector production involves transient transfection of one or more of these components into cells containing the other required components. The packaging cells of the present invention may be any mammalian cell type capable of producing retroviral/lentiviral vector particles. The packaging cells may be 293T-cells, or variants of 293T-cells which have been adapted to grow in suspension and grow without serum.

The packaging cells may be made by transient transfection with
a) the transfer vector
b) a gagpol expression vector
c) an env expression vector. The env gene may be a heterologous, resulting in a pseudotyped retroviral vector. For example, the env gene may be from RD1 14 or one of its variants, VSV-G, the Gibbon-ape leukaemia virus (GALV), the Amphotropic envolope or Measles envelope or baboon retroviral envelope glycoprotein.

In the case of lentiviral vector, transient transfection with a rev vector is also performed.

The present disclosure provides host cells expressing viral particles according to the foregoing embodiments. In some embodiments, the host cells express, at the cell surface, one or more transduction enhancers. In some embodiments, the present invention provides a host cell which expresses, at the cell surface,
(a) a mitogenic transduction enhancer comprising a mitogenic domain and a transmembrane domain; and/or
(b) a cytokine-based transduction enhancer which comprises a cytokine domain and a transmembrane domain;
such that a retroviral or lentiviral vector produced by the packaging cell is as described in the foregoing embodiments.

In some embodiments, the host cell may also express, at the cell surface, a tagging protein which comprises: a binding domain which binds to a capture moiety; and a transmembrane domain, which tagging protein facilitates purification of the viral vector from cellular supernatant via binding of the tagging protein to the capture moiety, such that a retroviral or lentiviral vector produced by the packaging cell has the characteristics describing in the foregoing sections.

The tagging protein may also comprise a spacer between the binding domain and the transmembrane domain.

The term host cell may be used to describe a packaging cell or a producer cell. A packaging cell may comprise one or more of the following genes: gag, pol, env and/or rev. A producer cell may comprise gag, pol, env and optionally rev genes and also comprises a retroviral or lentiviral genome. In some embodiments, the host cell may be any suitable cell line stably expressing mitogenic and/or cytokine transduction enhancers. It may be transiently transfected with transfer vector, gagpol, env (and rev in the case of a lentivirus) to produce replication incompetent retroviral/lentiviral vector.

The present disclosure also provides a method for making a host cell according to the above, which comprises the step of transducing or transfecting a cell with a nucleic acid encoding one or more transduction enhancers. Also provided is a method for producing a viral vector according to the foregoing embodiments which comprises the step of expressing a retroviral or lentiviral genome in a cell according to the second aspect of the invention.

### Systems and Kits

The present disclosure provides a system, therapeutic system, or composition, comprising:
(a) an adaptor molecule comprising a targeting moiety and a masked hapten, wherein the masked hapten comprises a masking moiety linked to a hapten; and
(b) a plurality of recombinant retroviral particles,
wherein each of the retroviral particles comprises a polynucleotide comprising, in 5′ to 3′ order:
(i) a 5′ long terminal repeat (LTR) or untranslated region (UTR),
(ii) a promoter,
(iii) a sequence encoding a receptor that specifically binds to the hapten, and
(iv) a 3′ LTR or UTR; and
wherein each of the retroviral particles comprises a viral envelope comprising
(i) a viral fusion glycoprotein, and
(ii) one or more transduction enhancers
wherein optionally each of the transduction enhancers is selected from the group consisting of a T-cell activation receptor, a NK-cell activation receptor, and a co-stimulatory molecule. In some embodiments, the polynucleotide additionally comprises a sequence encoding a T cell activator protein.

The present disclosure also provides a kit comprising the system and instructions for use of the system.

### Transgenic Immune Cells

The present disclosure provides a method for making an activated transgenic immune cell, which comprises the step of contacting an immune cell with a viral vector according to any of the foregoing embodiments. The immune cells may be transduced in vivo or ex vivo. In some embodiments, the viral vectors are administered to a living subject such that the immune cells are transduced in vivo without any need to isolate and manipulate host cells ex vivo. In some embodiments, immune cells are manipulated ex vivo and then returned to the subject in need thereof.

The immune cells generally are mammalian cells, and typically are human cells, more typically primary human cells, e.g., allogeneic or autologous donor cells. The cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immune systems, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+cells, CD8+cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+and/or of CD8+T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, herein, the cells provided are cytotoxic T lymphocytes. A “Cytotoxic T lymphocyte” (CTL) may include but is not limited to, for example, a T lymphocyte that expresses CD8 on the surface thereof (e.g., a CD8+T cell). In some embodiments, such cells are preferably “memory” T cells (TM cells) that are antigen-experienced. In some embodiments, the cell is a precursor T cell. In some embodiments, the precursor T cell is a hematopoietic stem cell. In some embodiments, the cell is a CD8+T cytotoxic lymphocyte cell selected from the group consisting of naive CD8+T cells, central memory CD8+T cells, effector memory CD8+T cells and bulk CD8+T cells. In some embodiments, the cell is a CD4+T helper lymphocyte cell that is selected from the group consisting of naive CD4+T cells, central memory CD4+T cells, effector memory CD4+T cells, and bulk CD4+T cells.

As used herein, any reference to a transgenic T cell or transduced T cell, or the use thereof, may also be applied to any of the other immune cell types disclosed herein.

The present disclosure also provides transgenic immune cells comprising one or more exogenous nucleic acid molecules. In some embodiments, the transgenic immune cells comprise polynucleotides encoding hapten-binding receptors. In some embodiments, the transgenic immune cells comprise polynucleotides encoding transduction enhancers. In some embodiments, the transgenic immune cells comprise polynucleotides encoding T cell activator proteins. In some embodiments, the transgenic immune cells comprise polynucleotides encoding hapten-binding receptors and polynucleotides encoding T cell activator proteins.

### Methods of Treating Subjects With the Disclosed Compositions

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed viral particles to the subject, wherein prior to, during, or after the administering step the subject received or receives a dose of an adaptor molecule comprising a targeting moiety and a masked hapten effective to label cancer cells with a hapten. Also provided is a method of treating a tumor and/or killing tumor cells in a subject, comprising administering an effective amount of an adaptor molecule to the subject, wherein: the adaptor molecule labels tumor cells with a masked hapten; wherein the masked hapten is activated by reactive oxygen species, generating a hapten; and wherein prior to, during, or after the administering step the subject received or received the retroviral particles according to any of the foregoing embodiments.

In some embodiments, a method disclosed herein may be use to treat cancer and/or kill cancer cells in a subject by administering a therapeutically effective amount of the lentiviral particles according to any of the foregoing embodiments, wherein prior to the administering step the subject has received a dose of adaptor molecule comprising a targeting moiety and a hapten, effective to label cancer cells with the hapten. In some embodiments, a method disclosed herein may be used to treat cancer and/or kill cancer cells by administering a system

The present disclosure also provides a method of treating cancer and/or killing cancer cells in a subject, comprising administering the system of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, the target cell is a tumor cell. In some embodiments, the target cell is an immune cell. In some embodiments, the immune cell is a T cell or a B cell. In some embodiments, the target cell exists in a tumor microenvironment.

In some embodiments, a transduced T cell is provided to the subject 1, 2, 3, 4, 5, 6, 7, 8, 9, 10, 11, 12, 15, 20, 24, 36, 48, 60 or 72 hours after administration of an adaptor molecule composition, or any time within a range defined by any two aforementioned values. In some embodiments, the cell is provided to the subject 1, 2, 3, 4, 5, 6, 7, 8, 9, 10, 11, 12, 15, 20, 24, 36 or 48 hours before administration of the composition, or any time within a range defined by any two aforementioned values. In some embodiments, the cell is provided to the subject within seconds or minutes, such as less than an hour, of providing the composition to the subject. In some embodiments, a boost of the cell and/or the composition is provided to the subject. In some embodiments, the viral vectors are administered directly to the subject. In some embodiments, viral vectors are administered in conjunction with T cells. In some embodiments, viral vectors and T cells are separately administered. In some embodiments, T cells are activated and transduced in vivo by administered viral vectors.

In some embodiments, an additional cancer therapy is provided, such as a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

### Modes of Administration and Dosing

The disclosed viral particles, adaptor molecules, and immune cells may be administered in a number of ways depending upon whether local or systemic treatment is desired.

In the case of adoptive cell therapy, methods for administration of cells for adoptive cell therapy are known and may be used in connection with the provided methods and compositions. For example, adoptive T cell therapy methods are described, e.g., in U.S. Patent Application Publication No. 2003/0170238 to Gruenberg et at; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In a preferred embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing immune cells or transgenic immune cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

In the context of administering viral particles, the amount of viral particles and time of administration of such particles will be within the purview of the skilled artisan having benefit of the present teachings. In some embodiments, the administration of therapeutically-effective amounts of the disclosed compositions may be achieved by a single administration, such as for example, a single injection of sufficient numbers of viral particles to provide therapeutic benefit to the patient undergoing such treatment. In some embodiments, the subject is provided multiple, or successive administrations of the lentiviral vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, viral particles/ml given either as a single dose, or divided into two or more administrations as may be required to achieve therapy of the particular disease or disorder being treated. In some embodiments, a subject may be administered two or more different viral vector compositions, either alone, or in combination with one or more other therapeutic drugs to achieve the desired effects of a particular therapy regimen. In some embodiments, the viral vectors are administered in combination with the transgenic immune cells. In some embodiments, the viral vectors are administered in combination with immune cells that have not yet been transduced. The phrase “in combination” may comprise at the same time or at different times within a short period of time, e.g., within one week, one day, twelve hours, six hours, one hour, thirty minutes, ten minutes, five minutes, or one minute.

In the context of administering adaptor molecules, the dose will depend on the type of target cell, the targeting moiety comprised by the adaptor molecule, and the hapten molecule comprised by the adaptor molecule. Depending on the type and severity of the disease, illustrative dosages for the adaptor molecules can range from about 1 μg/kg to about 50 mg/kg or from about 5 mg/kg to about 15 mg/kg, including but not limited to 5 mg/kg, 7.5 mg/kg, 10 mg/kg or 15 mg/kg. The frequency of administration will vary depending on the type and severity of the disease. For repeated administrations over several days or longer, depending on the condition, the treatment may be sustained until the condition, e.g., cancer, is treated or the desired therapeutic effect is achieved, as measured by methods known in the art. In some embodiments, the adaptor molecules are administered one time. In some embodiments, the adaptor molecules are administered once every 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 1 year. The adaptor molecules may be administered in combination with the viral particles and/or transgenic immune cells disclosed herein. The phrase “in combination” may comprise at the same time or at different times within a short period of time, e.g., within one week, one day, twelve hours, six hours, one hour, thirty minutes, ten minutes, five minutes, or one minute.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarding as the invention.

### Example 1: FITC CAR Transduction, Enrichment, and Activation

This example demonstrates enhanced expansion and activation of CAR-expressing T cells in the presence of the antigen fluorescein isothiocyanate (FITC).

### Thawing and Resting PBMCs

On Day 0 of the protocol, frozen peripheral blood mononuclear cells (PBMCs) were quickly thawed in 37° C. bath water and slowly added to 9 mL of warm RPMI-C (RPMI 1640+P/S+10% FBS) media. The thawed PBMCs were then spun at 200×g for 10 min. Following the spin, the media was aspirated, the cells were counted and plated in RPMI-C media at a cell density of about 1×10⁶ cells/mL in a 250 mL flask. 50 U/mL IL-2 was also added to the flask. After plating, the total live cell count was 2.32×10⁷ cells in 20 mL of IL-2 containing RPMI-C media.

### Transduction With FITC CAR Vector

On day 1 of the protocol, 1×10⁷ PBMC cells were collected in a 50 mL conical tube and spun at 400×g for 5 min. The cells were then plated at a concentration of 2.5×10⁶ cells/well in a total of 2.5 mL of RPMI-C media with 50 U/mL IL-2 in a 6-well plate. Lentiviral particles engineered with a CD3-Cocal envelope and containing a FITC CAR-Frb-RACR multicistronic vector were used to transduce the PBMCs at a multiplicity of infection (MOI) of 10. The lentiviral titer used for the transduction was 1.61×10⁸ TU/mL, with a target titer of 2.5×10⁷ TU/well. The volume of particles added to each well was 155 μL. The transduced cells remained in the incubator for 6 days and 50 U/mL IL-2 was added to the cells every 3 days. The transduced cells were monitored daily and 50 U/mL IL-2 containing RPMI-C media was added to a well if the cell media was observed to develop an orange-yellow coloration.

### Split Cells Into Rapamycin

On day 7 of the protocol, 1×10⁷ PBMC cells were collected into 15 mL conical tubes, washed with RPMI-C media, and spun at 400×g for 5 min. The cells were split into 2 wells of a 6-well plate at a cell density of 2×10⁶ cells/well in 2 mL RPMI-C media with 50 U/mL IL-2. 10 nM rapamycin was added to one of the wells and 0 nM was added to the second well. The rapamycin treated and untreated cells remained in the incubator for 4 days and 50 U/mL IL-2 was added to the cells every 3 days. The cells were monitored daily and 50 U/mL IL-2 containing RPMI-C media +/−10 nM rapamycin was added to a well if the cell media was observed to develop an orange-yellow coloration.

### Coat Plate With FITC-OVA

On day 10 of the protocol, 5 μg/mL fluorescently labelled ovalbumin (FITC-OVA) solution in PBS was prepared. 125 μL of the FITC-OVA solution was added per well of a 48-well plate. The plated was incubated with the solution overnight at 4° C. wrapped in aluminum foil.

### FITC-Biotin Bead Conjugation

On day 10 of the protocol, anti-biotin MACSiBead particles were vortexed and 30 μg of FITC-biotin primary antibody were added to 10×10⁸ MACSiBead particles. The total volume was brought to 1 mL using a buffer containing PBS with 0.5% BSA and 2 nM EDTA.

### Split Cells Into Antigen (FITC) Stimulation Conditions

On day 11 of the protocol, 4.8×10⁶ total live cells that were incubated in 0 nM and 1.9×10⁶ total live cells that were incubated in 10 nM rapamycin were collected into 15 mL conical tubes, washed with RPMI-C media, and spun at 400×g for 5 min. The washed cells were resuspended at a cell density of 2.5×10⁶ cells per mL in RPMI-C media without IL-2.

The FITC-OVA solution was aspirated from the treated plates and washed three times with cold PBS. The cells were divided into 48-well plates, 2.5×10⁵ cells/well in to 500 μL RPMI-C media +/−10 nM rapamycin. Table 1 depicts the stimulation conditions of the samples. For cells treated under FITC-biotin bead conditions, a 1:1 ratio of beads: cells was used. The cells remained in the incubator for 5 days. The cells were monitored daily and RPMI-C media +/−10 nM rapamycin was added to a well if the cell media was observed to develop an orange-yellow coloration.

TABLE 1

| Unstimulated | | FITC-biotin beads | | Platebound FITC-OVA | |
|---|---|---|---|---|---|
| 0 nM | 10 nM | 0 nM | 10 nM | 0 nM | 10 nM |
| rapamycin | rapamycin | rapamycin | rapamycin | rapamycin | rapamycin |

### Assessment of CAR Expression and Activation by Flow Cytometry

On day 16 of the protocol, the cells in each of the stimulation conditions were counted and the results of the cell count are depicted in Table 2.

TABLE 2

| | | FITC-biotin | Platebound |
|---|---|---|---|
| Condition | Unstimulated | beads | FITC-OVA |
| PBMC FITC | 1 × 10⁵ cells | 1 × 10⁵ cells | 2.6 × 10⁵ cells |
| CAR-Frb-RACR | | | |
| 0 nM rapamycin | | | |
| PBMC FITC | 2.4 × 10⁵ cells | 2.6 × 10⁵ cells | 5.2 × 10⁵ cells |
| CAR-Frb-RACR | | | |
| 10 nM rapamycin | | | |

Cells were transferred to individual wells of a 96-well V-bottom plate, the plate was spun at 400×g for 5 min, cells were washed with 200 μL PBS and spun again at 400×g for 5 min. Cells were incubated for 10 min at room temperature in 50 μL/well Zombie NIR Fixable Viability Dye (diluted 1:3000 in PBS). Cells were then washed with 150 μFACS buffer (1×PBS+2% FBS) and the plate was spun at 400×g for 5 min. Next, 50 μL/well of surface stain in FACS buffer were added to the cells and incubated for 45 min at 4° C.

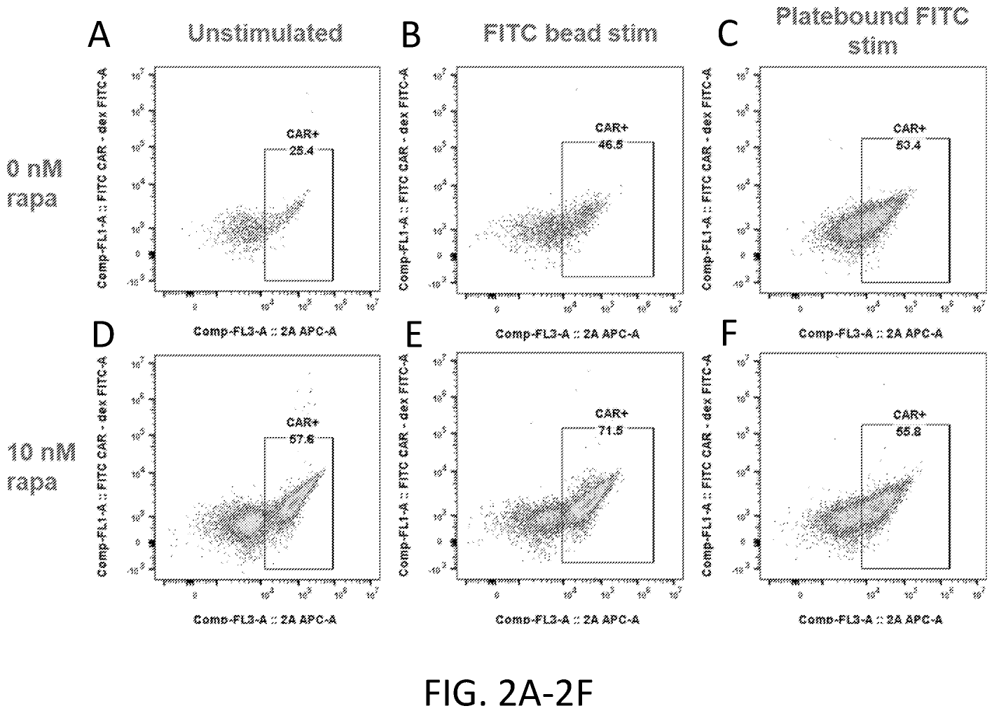

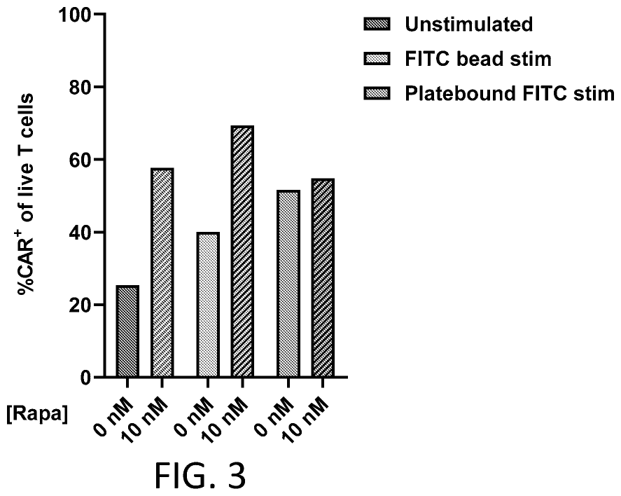

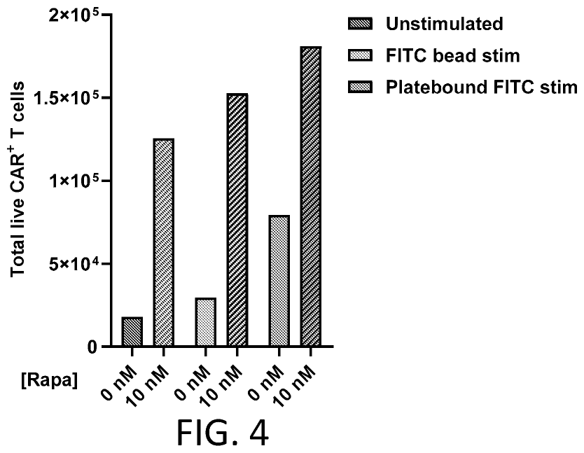

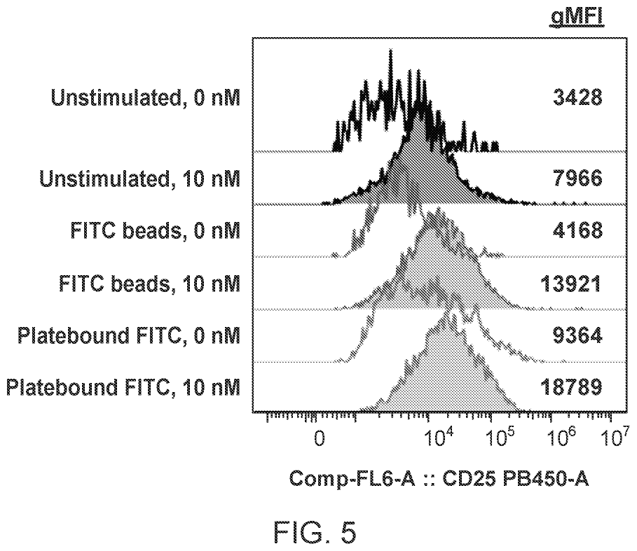

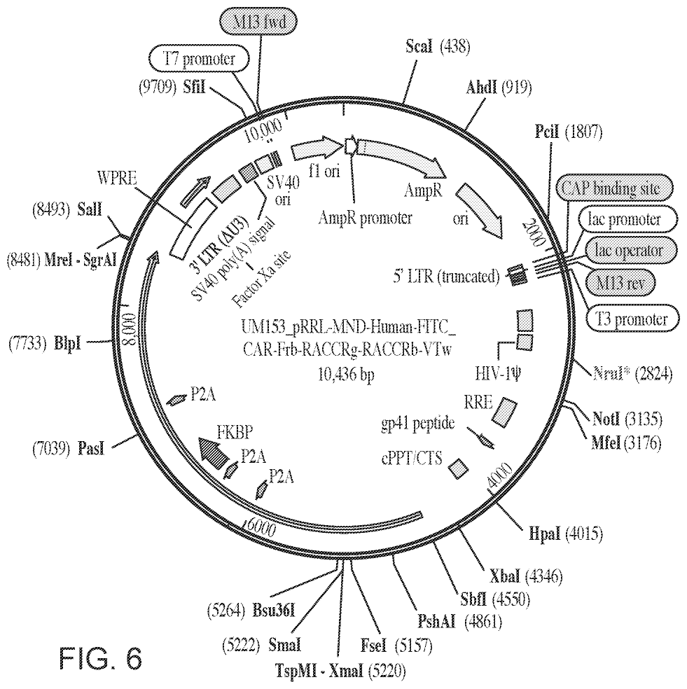

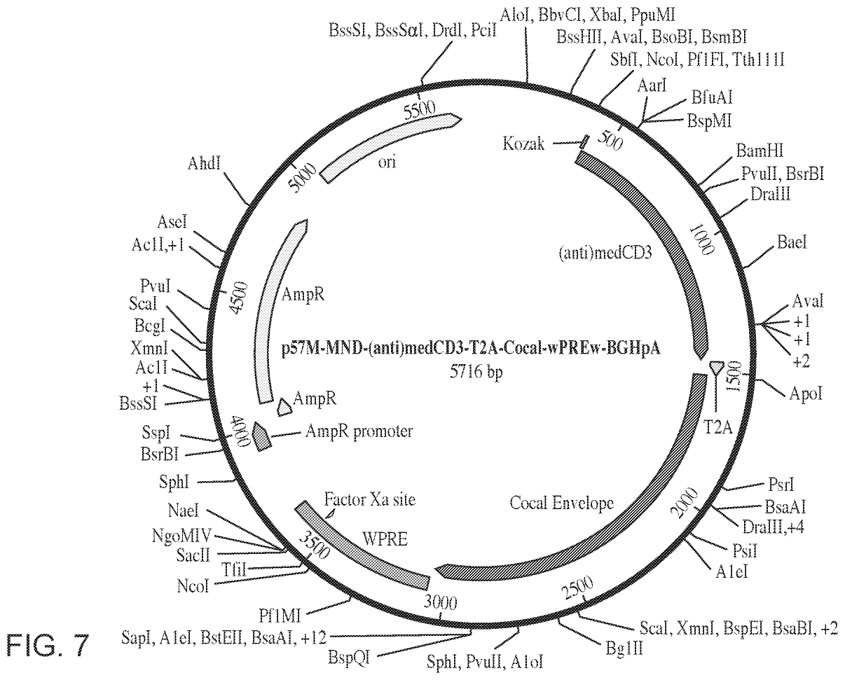

Cells were washed with 150 μL FACS buffer and the plate was spun at 400×g for 5 min. Cells were then fixed with 100 μL/well of BD Cytofix/Cytoperm buffer for 20 min at 4° C. Cells were then washed with 100 μL BD Perm/Wash diluted 10-fold in water and the plate was spun at 400×g for 5 min. Cells were washed again with 200 μL BD Perm/Wash diluted 10-fold in water and the plate was spun again at 400×g for 5 min. 50 μL/well of intracellular stain in 1×BD Perm/Wash was added to the cells and incubated for 30 min at 4° C. Cells were washed with 150 μL, FACS buffer, the plate was spun at 400×g for 5 min and the cells were resuspended in 200 μL FACS buffer for analysis using a Beckman Coulter CytoFLEX S cytometer. As depicted in FIGS. 2A-2F, CAR-expressing T cells expand in the presence of the antigen FITC and this expansion is enhanced with rapamycin.

Antibodies and fluorescently labelled molecules for flow cytometric analysis were used at the following dilutions:
FITC dextran (1:10)
CD3-AF700 (1:100)
CD25-BV421 (1:100)
PD1-BV650 (1:100)
Lag3-PECy7 (1:100)
2A-AF647 (1:100)

1. A method, comprising:
(a) administering to a subject an adaptor molecule comprising a targeting moiety and a hapten; and
(b) administering to the subject either (i) a plurality of recombinant retroviral particles or (ii) immune cells that have been contacted ex vivo with a plurality of recombinant retroviral particles,
wherein each of the retroviral particles comprises a polynucleotide comprising a sequence encoding a receptor that specifically binds to the hapten; and
wherein each of the retroviral particles comprises a viral envelope.

2. The method of claim 1, wherein the immune cells are T cells.

3. The method of claim 1 or 2, wherein the retroviral particles are lentiviral particles.

4. The method of any one of claims 1-3, wherein the viral envelope comprises a cell-surface receptor that specifically binds to the immune cell.

5. The method of claim 4, wherein the cell-surface receptor comprises a multipartite signaling complex.

6. The method of claim 5, wherein the multipartite signaling complex forms a functional multipartite signaling complex in the presence of a bridging factor.

7. The method of any one of claims 1-6, wherein the viral envelope comprises one or more transduction enhancers.

8. The method of claim 7, wherein each of the transduction enhancers is a T-cell activation receptor, an NK-cell activation receptor, or a co-stimulatory molecule.

9. The method any one of claims 1-8, wherein the adaptor molecule comprises a masked hapten comprising one or more masking moieties covalently linked to the hapten.

10. The method of claim 7, wherein the masked hapten is configured to permit a chemical reaction to remove the masking moiety from the hapten.

11. The method of any claim 9 or 10, wherein the masked hapten is configured to permit reactive oxygen species to remove the masking moiety from the hapten.

12. The method of any one of claims 9-11, wherein the masked hapten comprises a hydroxyphenyl group.

13. The method of any one of claims 9-12, wherein the hapten comprises a 2,4-dinitrophenol (DNP) group.

14. The method of any one of claims 1-13, wherein the hapten comprises a fluorescein.

15. The method of claim 14, wherein the hapten is a masked hapten comprising hydroxyphenyl fluorescein (HPF).

16. The method of claim 14, wherein the hapten is a masked hapten comprising fluorescein-DNP.

17. The method of any one of claims 1-16, wherein the targeting moiety comprises a phospho-lipid ether (PLE).

18. The method of any one of claims 1-16, wherein the targeting moiety comprises a folate.

19. The method of claim 15, wherein the adaptor molecule comprises HPF conjugated to a PLE.

20. The method of claim 15, wherein the adaptor molecule comprises a HPF-PEG₃-C₁₈-alkylphospholipid.

21. The method of claim 17, wherein the adaptor molecule comprises the molecule of formula I:

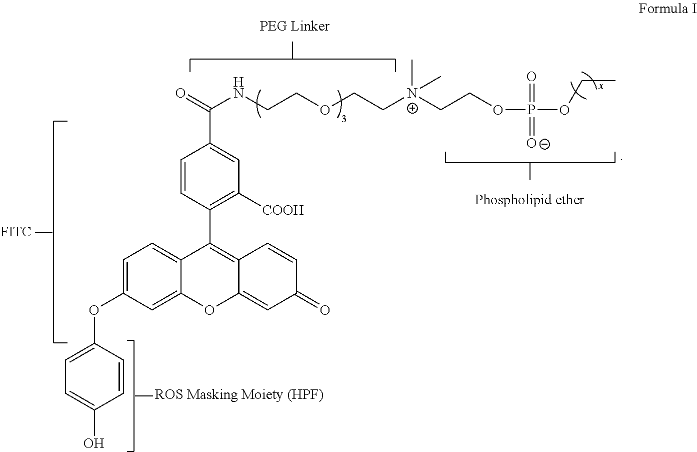

22. The method of claim 17, wherein the adaptor molecule comprises a HPF-{linker}-erufosine.

23. The method of any one of claims 1-22, wherein the viral envelope comprises a viral fusion glycoprotein from the Cocal strain or a functional variant thereof.

24. The method of any one of claims 1-23, wherein the viral envelope comprises a viral fusion glycoprotein comprising an amino acid sequence at least 95% identical to SEQ ID NO: 1 (Cocal G protein).

25. The method of any one of claims 1-24, wherein the one or more transduction enhancers comprise one or more of anti-CD3scFv, CD86, and CD137L

26. The method of any one of claims 1-25, wherein the transduction enhancers comprise every one of anti-CD3scFv, CD86, and CD137L.

27. The method of any one of claims 1-26, wherein the polynucleotide comprises a sequence encoding at least one T-cell activator protein.

28. The method of claim 27, wherein the at least one T-cell activator protein is a dimeric T-cell activator receptor.

29. The method of claim 27 or 28, wherein the at least one T-cell activator protein comprises a first receptor protein comprising a first dimerization domain and a second receptor protein comprising a second dimerization domain,
wherein the first dimerization domain and the second dimerization domain specifically bind to one another in response to a molecule.

30. The method of claim 29, wherein the molecule is selected from the list consisting of: FK1012, tacrolimus (FK506), FKCsA, rapamycin, coumermycin, gibberellin, HaXS, TMP-HTag, ABT-737, and functional derivatives thereof.

31. The method of any one of claims 1-30, wherein the receptor that specifically binds to the hapten comprises a hapten-specific antigen-binding fragment of an antibody.

32. The method of claim 31, wherein the antigen-binding fragment comprises an Fab fragment, a single-chain Fv fragment (scFv) or a single heavy chain antibody.

33. The method of any one of claims 1-32, wherein the receptor that specifically binds to the hapten comprises a hapten-specific chimeric antigen receptor.

34. The method of claim 33, wherein the hapten-specific chimeric antigen receptor comprises an amino acid sequence at least 95% identical to the amino acid sequence hapten: NN.

35. The method any one of claims 1-34, wherein T cells in the subject are transduced by the retroviral particles.

36. The method any one of claims 1-35, wherein T cells in the subject express a receptor that specifically binds to the hapten.

37. The method any one of claims 1-36, wherein the adaptor molecule specifically binds to and/or labels cancer cells in the subject.

38. The method any one of claims 1-37, wherein the masked hapten is removed by a chemical reaction in the subject.

39. The method any one of claims 1-38, wherein T cells transduced by the retroviral particles specifically kill cancer cells comprising an unmasked hapten.

40. The method any one of claims 1-39, wherein the subject suffers from a cancer and the method treats the cancer.

41. The method any one of claims 1-40, wherein the method kills tumor cells.

42. A system, therapeutic system, or composition, comprising:
(a) an adaptor molecule comprising a targeting moiety and a masked hapten, wherein the masked hapten comprises a masking moiety linked to a hapten; and
(b) a plurality of recombinant retroviral particles,
wherein each of the retroviral particles comprises a polynucleotide comprising, in 5′ to 3′ order:
(i) a 5′ UTR,
(ii) a promoter,
(iii) a sequence encoding a receptor that specifically binds to the hapten, and
(iv) a 3′ UTR;
and
wherein each of the retroviral particles comprises a viral envelope comprising
(i) a cell-surface receptor, and
(ii) one or more transduction enhancers
wherein optionally each of the transduction enhancers is selected from the group consisting of a T-cell activation receptor, a NK-cell activation receptor, and a co-stimulatory molecule.

43. The system of claim 42, wherein the retroviral particles are lentiviral particles.

44. The system of claim 42 or 43, wherein the viral envelope comprises a cell-surface receptor that specifically binds to the immune cell.

45. The system of claim 44, wherein the cell-surface receptor comprises a multipartite signaling complex.

46. The system of claim 45, wherein the multipartite signaling complex forms a functional multipartite signaling complex in the presence of a bridging factor.

47. The system of any one of claims 42-46, wherein the viral envelope comprises one or more transduction enhancers.

48. The system of claim 47, wherein each of the transduction enhancers is a T-cell activation receptor, an NK-cell activation receptor, or a co-stimulatory molecule.

49. The system any one of claims 42-48, wherein the adaptor molecule comprises a masked hapten comprising one or more masking moieties covalently linked to the hapten.

50. The system of claim 49, wherein the masked hapten is configured to permit a chemical reaction to remove the masking moiety from the hapten.

51. The system of any claim 49 or 50, wherein the masked hapten is configured to permit reactive oxygen species to remove the masking moiety from the hapten.

52. The system of any one of claims 49-51, wherein the masked hapten comprises a hydroxyphenyl group.

53. The system of any one of claims 49-52, wherein the masking moiety comprises a 2,4-dinitrophenol (DNP) group.

54. The system of any one of claims 42-53, wherein the hapten comprises a fluorescein.

55. The system of claim 54, wherein the hapten is a masked hapten comprising hydroxyphenyl fluorescein (HPF).

56. The system of claim 54, wherein the hapten is a masked hapten comprising fluorescein-DNP.

57. The system of any one of claims 42-56, wherein the targeting moiety comprises a phospho-lipid ether (PLE).

58. The system of any one of claims 42-56, wherein the targeting moiety comprises a folate.

59. The system of claim 57, wherein the adaptor molecule comprises HPF conjugated to a PLE.

60. The system of claim 57, wherein the adaptor molecule comprises a HPF-FITC-PEG₃-C₁₈-alkylphospholipid.

61. The system of claim 49, wherein the adaptor molecule comprises the molecule of formula I:

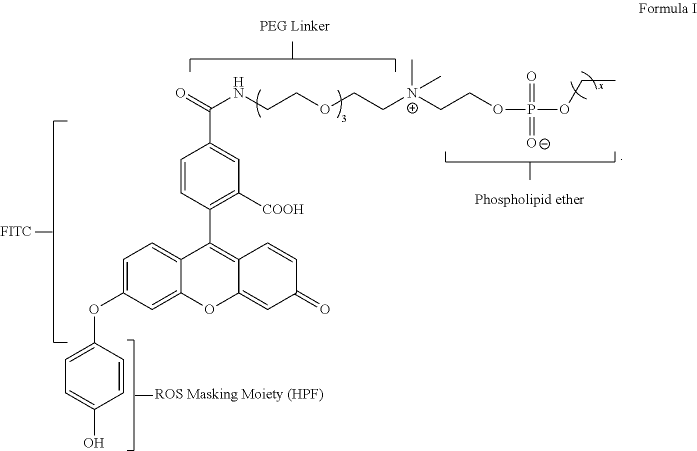

62. The system of claim 57, wherein the adaptor molecule comprises a HPF-{linker}-erufosine.

63. The system of any one of claims 42-62, wherein the viral envelope comprises a viral fusion glycoprotein from the Cocal strain or a functional variant thereof.

64. The system of any one of claims 42-63, wherein the viral envelope comprises a viral fusion glycoprotein comprising an amino acid sequence at least 95% identical to SEQ ID NO: 1 (Cocal G protein).

65. The system of any one of claims 42-64, wherein the one or more transduction enhancers comprise one or more of anti-CD3scFv, CD86, and CD137L

66. The system of any one of claims 42-65, wherein the transduction enhancers comprise every one of anti-CD3scFv, CD86, and CD137L.

67. The system of any one of claims 42-66, wherein the polynucleotide comprises a sequence encoding at least one T-cell activator protein.

68. The system of claim 67, wherein the at least one T-cell activator protein is a dimeric T-cell activator receptor.

69. The system of claim 67 or 68, wherein the at least one T-cell activator protein comprises a first receptor protein comprising a first dimerization domain and a second receptor protein comprising a second dimerization domain,
wherein the first dimerization domain and the second dimerization domain specifically bind to one another in response to a molecule.

70. The system of claim 69, wherein the molecule is selected from the list consisting of: FK1012, tacrolimus (FK506), FKCsA, rapamycin, coumermycin, gibberellin, HaXS, TMP-HTag, ABT-737, and functional derivatives thereof.

71. The system of any one of claims 42-70, wherein the receptor that specifically binds to the hapten comprises a hapten-specific antigen-binding fragment of an antibody.

72. The system of claim 71, wherein the antigen-binding fragment comprises an Fab fragment, a single-chain Fv fragment (scFv), or a single heavy chain antibody.

73. The system of any one of claims 42-72, wherein the receptor that specifically binds to the hapten comprises a hapten-specific chimeric antigen receptor.

74. The system of claim 73, wherein the hapten-specific chimeric antigen receptor comprises an amino acid sequence at least 95% identical to the amino acid sequence SEQ ID NO: 53.

75. The system any one of claims 42-74, wherein T cells in the subject are transduced by the retroviral particles.

76. The system any one of claims 42-75, wherein T cells in the subject express a receptor that specifically binds to the hapten.

77. The system any one of claims 42-76, wherein the adaptor molecule specifically binds to and/or labels cancer cells in the subject.

78. The system any one of claims 42-77, wherein the masked hapten is removed by a chemical reaction in the subject.

79. The system any one of claims 42-78, wherein T cells transduced by the retroviral particles specifically kill cancer cells comprising an unmasked hapten.

80. The system any one of claims 42-79, wherein the subject suffers from a cancer and the system treats the cancer.

81. The system any one of claims 42-80, wherein the system kills tumor cells.

82. A kit, comprising the system of any one of claims 42-81 and instructions for use of the system.

83. A retroviral particle, comprising
(a) a polynucleotide comprising, in 5′ to 3′ order:
(i) a 5′ LTR or UTR,
(ii) a promoter,
(iii) a sequence encoding a receptor that specifically binds to the hapten, and
(iv) a 3′ LTR or UTR; and
(b) a viral envelope comprising
(i) a cell-surface receptor, and
(ii) one or more transduction enhancers; and
wherein optionally each of the transduction enhancers is a T-cell activation receptor, a NK-cell activation receptor, or a co-stimulatory molecule.

84. A therapeutic composition comprising an amount of the retroviral particle of claim 83 sufficient to cause cancer cell death in a subject to which an adaptor molecule comprising targeting moiety and a masked hapten has been, is, or will be administered.

85. A method of treating cancer and/or killing cancer cells in a subject, comprising administering a therapeutically effective amount of the retroviral particle of claim 83 to the subject, wherein prior to, during, or after the administering step the subject received or receives a dose of an adaptor molecule comprising a targeting moiety and a masked hapten effective to label cancer cells with a hapten.

86. A method of treating a tumor and/or killing tumor cells in a subject, comprising administering an effective amount of an adaptor molecule to the subject, wherein:
wherein the adaptor molecule labels tumor cells with a masked hapten;
wherein the masked hapten is activated by reactive oxygen species, generating a hapten; and
wherein prior to, during, or after the administering step the subject received or received the retroviral particle of claim 83.

87. A method of treating cancer and/or killing cancer cells in a subject, comprising administering a therapeutically effective amount of the retroviral particle of claim 83 to the subject, wherein prior to the administering step the subject has received a dose of targeting moiety and a masked hapten effective to label cancer cells with the hapten.

88. A cell line configured to produce the retroviral particle of claim 83.

89. A method of treating cancer and/or killing cancer cells in a subject, comprising administering the system of any one of claims 42-81 to the subject.


SEQUENCE LISTING (53 sequences)

SEQ ID NO: 1 - protein, 511 aa, Artificial Sequence
  Cocal G protein
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 2-3 - skipped (2 entries)

SEQ ID NO: 4 - protein, 7 aa, Artificial Sequence
  OKT3 CDRH1
GYTFTRY

SEQ ID NO: 5 - protein, 6 aa, Artificial Sequence
  OKT3 CDRH2
NPSRGY

SEQ ID NO: 6 - protein, 10 aa, Artificial Sequence
  OKT3 CDRH3
YYDDHYCLDY

SEQ ID NO: 7 - protein, 10 aa, Artificial Sequence
  OKT3 CDRL1
SASSSVSYMN

SEQ ID NO: 8 - protein, 7 aa, Artificial Sequence
  OKT3 CDRL2
DTSKLAS

SEQ ID NO: 9 - protein, 9 aa, Artificial Sequence
  OKT3 CDRL3
QQWSSNPFT

SEQ ID NO: 10 - protein, 7 aa, Artificial Sequence
  CD28 antibody - CDRH1
GFSLTSY

SEQ ID NO: 11 - protein, 5 aa, Artificial Sequence
  CD28 antibody - CDRH2
WAGGS

SEQ ID NO: 12 - protein, 15 aa, Artificial Sequence
  CD28 antibody - CDRH3
DKRAPGKLYYGYPDY

SEQ ID NO: 13 - protein, 15 aa, Artificial Sequence
  CD28 Antibody - CDRL1
RASESVEYYVTSLMQ

SEQ ID NO: 14 - protein, 7 aa, Artificial Sequence
  CD28 antibody - CDRL2
AASNVES

SEQ ID NO: 15 - protein, 9 aa, Artificial Sequence
  CD28 antibody - CDRL3
QQTRKVPST

SEQ ID NO: 16 - protein, 6 aa, Artificial Sequence
  TGN1412 - CDRH1
GYTFSY

SEQ ID NO: 17 - protein, 6 aa, Artificial Sequence
  TGN1412 - CDRH2
YPGNVN

SEQ ID NO: 18 - protein, 11 aa, Artificial Sequence
  TGN1412 - CDRH3
SHYGLDWNFDV

SEQ ID NO: 19 - protein, 10 aa, Artificial Sequence
  TGN1412 - CDRL1
HASQNIYVLN

SEQ ID NO: 20 - protein, 7 aa, Artificial Sequence
  TGN1412 - CDRL2
KASNLHT

SEQ ID NO: 21 - protein, 9 aa, Artificial Sequence
  TGN1412 - CDRL3
QQGQTYPYT

SEQ ID NO: 22 - protein, 173 aa, Artificial Sequence
  OX40L sequence
MERVQPLEENVGNAARPRFERNKLLLVASVIQGLGLLLCFTYICLHFSALQVSHRYPRIQSIKVQFTEYKKEKGFILTSQKEDEIMKVQNYLISLKGYFSQEVNISLHYQKDEEPLFQLKKVRSVNSLMVASLTYKDKVYLNVTTDNTSLDDFHVNGGELILIHQNPGEFCVL

SEQ ID NO: 23 - protein, 254 aa, Artificial Sequence
  4-1BBL sequence
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 24 - protein, 233 aa, Artificial Sequence
  hinge-CH2CH3 of human lgG1
AEPKSPDKTHTCPPCPAPPVAGPSVFLFPPKPKDTLMIARTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 25 - protein, 46 aa, Artificial Sequence
  human CD8 stalk
TTTPAPRPPTPAPTIASQPLSLRPEACRPAAGGAVHTRGLDFACDI

SEQ ID NO: 26 - protein, 20 aa, Artificial Sequence
  human lgG1 hinge
AEPKSPDKTHTCPPCPKDPK

SEQ ID NO: 27 - protein, 185 aa, Artificial Sequence
  CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 28 - protein, 259 aa, Artificial Sequence
  CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 29 - protein, 153 aa, Artificial Sequence
  IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 177 aa, Artificial Sequence
  IL7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 31 - protein, 162 aa, Artificial Sequence
  IL15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 271 aa, Artificial Sequence
  membrane-IL7
MAHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 33 - protein, 256 aa, Artificial Sequence
  membrane-IL15
MGLVRRGARAGPRMPRGWTALCLLSLLPSGFMAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 34 - protein, 251 aa, Artificial Sequence
  IL2Ry complex
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 35 - skipped

SEQ ID NO: 36 - protein, 251 aa, Artificial Sequence; identical to SEQ ID NO: 34
  IL2Ry complex

SEQ ID NO: 37 - protein, 251 aa, Artificial Sequence; identical to SEQ ID NO: 34
  IL2Ry complex

SEQ ID NO: 38 - protein, 251 aa, Artificial Sequence; identical to SEQ ID NO: 34
  IL2Ry complex

SEQ ID NO: 39 - protein, 429 aa, Artificial Sequence
  IL2Rb complex
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 40 - protein, 429 aa, Artificial Sequence; identical to SEQ ID NO: 39
  IL2Rb complex

SEQ ID NO: 41 - protein, 429 aa, Artificial Sequence; identical to SEQ ID NO: 39
  IL2Rb complex

SEQ ID NO: 42 - protein, 429 aa, Artificial Sequence; identical to SEQ ID NO: 39
  IL2Rb complex

SEQ ID NO: 43 - protein, 429 aa, Artificial Sequence; identical to SEQ ID NO: 39
  IL7Ra complex

SEQ ID NO: 44 - protein, 4 aa, Artificial Sequence
  glycine spacer
GGGS

SEQ ID NO: 45 - protein, 7 aa, Artificial Sequence
  glycine spacer
GGGSGGG

SEQ ID NO: 46 - protein, 3 aa, Artificial Sequence
  glycine spacer
GGG

SEQ ID NO: 47 - protein, 251 aa, Artificial Sequence; identical to SEQ ID NO: 34
  IL2Ry complex

SEQ ID NO: 48 - protein, 429 aa, Artificial Sequence; identical to SEQ ID NO: 39
  IL2Rb complex

SEQ ID NO: 49 - protein, 429 aa, Artificial Sequence; identical to SEQ ID NO: 39
  IL2Ra complex

SEQ ID NO: 50 - protein, 251 aa, Artificial Sequence; identical to SEQ ID NO: 34
  IL7Ra complex

SEQ ID NO: 51 - protein, 20 aa, Artificial Sequence
  2a-like self-cleaving peptide
RAEGRGSLLTCGDVEENPGP

SEQ ID NO: 52 - protein, 20 aa, Artificial Sequence
  2a-like self-cleaving peptide
QCTNYALLKLAGDVESNPGP

SEQ ID NO: 53 - protein, 448 aa, Artificial Sequence
  FITC-CAR
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR